SEQUENCE LISTING (13 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 362 aa, Homo sapiens
MATAETALPSISTLTALGPFPDTQDDFLKWWRSEEAQDMGPGPPDPTEPPLHVKSEDQPGEEEDDERGADATWDLDLLLTNFSGPEPGGAPQTCALAPSEASGAQYPPPPETLGAYAGGPGLVAGLLGSEDHSGWVRPALRARAPDAFVGPALAPAPAPEPKALALQPVYPGPGAGSSGGYFPRTGLSVPAASGAPYGLLSGYPAMYPAPQYQGHFQLFRGLQGPAPGPATSPSFLSCLGPGTVGTGLGGTAEDPGVIAETAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 3 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 4 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 5 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 6 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 7 - protein, 464 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 8 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 9 - protein, 391 aa, Homo sapiens
MYSMMMETDLHSPGGAQAPTNLSGPAGAGGGGGGGGGGGGGGGAKANQDRVKRPMNAFMVWSRGQRRKMAQENPKMHNSEISKRLGAEWKVMSEAEKRPFIDEAKRLRALHMKEHPDYKYRPRRKTKTLLKKDKYSLAGGLLAAGAGGGGAAVAMGVGVGVGAAAVGQRLESPGGAAGGGYAHVNGWANGAYPGSVAAAAAAAAMMQEAQLAYGQHPGAGGAHPHAHPAHPHPHHPHAHPHNPQPMHRYDMGALQYSPISNSQGYMSASPSGYGGLPYGAAAAAAAAAGGAHQNSAVAAAAAAAAASSGALGALGSLVKSEPSGSPPAPAHSRAPCPGDLREMISMYLPAGEGGDPAAAAAAAAQSRLHSLPQHYQGAGAGVNGTVPLTHI

SEQ ID NO: 10 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 11 - protein, 446 aa, Homo sapiens
MRPVRENSSGARSPRVPADLARSILISLPFPPDSLAHRPPSSAPTESQGLFTVAAPAPGAPSPPATLAHLLPAPAMYSLLETELKNPVGTPTQAAGTGGPAAPGGAGKSSANAAGGANSGGGSSGGASGGGGGTDQDRVKRPMNAFMVWSRGQRRKMALENPKMHNSEISKRLGADWKLLTDAEKRPFIDEAKRLRAVHMKEYPDYKYRPRRKTKTLLKKDKYSLPSGLLPPGAAAAAAAAAAAAAAASSPVGVGQRLDTYTHVNGWANGAYSLVQEQLGYAQPPSMSSPPPPPALPPMHRYDMAGLQYSPMMPPGAQSYMNVAAAAAAASGYGGMAPSATAAAAAAYGQQPATAAAAAAAAAAMSLGPMGSVVKSEPSSPPPAIASHSQRACLGDLRDMISMYLPPGGDAADAASPLPGGRLHGVHQHYQGAGTAVNGTVPLTHI

SEQ ID NO: 12 - protein, 233 aa, Homo sapiens [sequence omitted]

SEQ ID NO: 13 - protein, 384 aa, Homo sapiens [sequence omitted]

(19) United States
(12) Patent Application Publication
(10) Pub. No.: US 2025/0145965 A1
(43) Pub. Date: May 8, 2025

(54) PRODUCTION METHOD FOR INDUCED PLURIPOTENT STEM CELLS

(71) Applicants: RIKEN, Wako-shi, Saitama (JP); KANEKA CORPORATION, Osaka-shi, Osaka (JP)

(72) Inventors: Yohei HAYASHI, Tsukuba-shi (JP); Mami TAKASAKI, Tsukuba-shi (JP); Sho KAMBAYASHI, Kobe-shi (JP); Yoshikazu KAWAI, Kobe-shi (JP)

(73) Assignees: RIKEN, Wako-shi, Saitama (JP); KANEKA CORPORATION, Osaka-shi, Osaka (JP)

(21) Appl. No.: 18/835,550

(22) PCT Filed: Feb. 2, 2023
(86) PCT No.: PCT/JP2023/003333

§ 371 Date: Aug. 2, 2024

(30) Foreign Application Priority Data
Feb. 4, 2022 (JP) 2022-016513

(51) Int. Cl. C12N 5/074; C12N 15/86

(52) U.S. Cl. CPC C12N 5/0696; C12N 15/86; C12N 2501/727; C12N 2510/00; C12N 2760/18843

(57) ABSTRACT

To provide a method for producing induced pluripotent stem cells (iPS cells) that can initialize somatic cells without using feeder cells or a substrate. For production of iPS cells, the following steps are carried out: I. introducing an initialization gene into a somatic cell; and II. performing initialization and amplification culture of the cell into which the gene has been introduced, in a liquid medium comprising at least one of a protein kinase Cβ (PKCβ) inhibitor and a WNT inhibitor under a suspension culture condition.

### TECHNICAL FIELD

The present invention relates to a method for producing induced pluripotent stem cells (iPS cells) under a suspension culture condition.

### BACKGROUND ART

Pluripotent stem cells such as induced pluripotent stem cell (iPS cells: induced pluripotent stem cells) have an ability to permanently proliferate and an ability to differentiate into various somatic cells. Putting therapeutic methods to transplant somatic cells obtained by inducing differentiation of pluripotent stem cells into practical use possibly leads to fundamental revolution for therapeutic methods for refractory diseases and lifestyle diseases. For example, techniques to induce pluripotent stem cells in vitro to differentiate into nerve cells and a wide variety of somatic cells including cardiomyocytes, blood cells, and retinal cells have been already developed. In addition, attempts have been made to prepare a stock of HLA homo-type iPS cells that are less likely to cause immune rejection in many people when differentiation-induced somatic cells are transplanted; and recently, in order to minimize the risk of immune rejection or the like upon transplantation into patients, attempts also have been made to prepare iPS cells of each patient.

Cell initialization techniques represented by a production technique of iPS cells have been rapidly progressed as innovative basic techniques in the life science, drug discovery and regenerative medicine. iPS cells are induced by introducing initialization factors such as OCT3/4, SOX2, KLF4, C-MYC and the like into somatic cells (Patent Literatures 1 and 2, and Non-Patent Literatures 1 and 2). All of these initialization factors are considered to work as transcription factors to regulate the expression of a group of genes involved in self-replication or pluripotency, thereby inducing initialization of somatic cells. Note that iPS cells are not induced only by introducing these initialization factors into somatic cells but iPS cells are gradually induced also by a certain period of culture after introduction of an initialization factor and establishment of iPS cells is completed. That is, the quality and purity of iPS cell population to be established are significantly affected not only by the purity or the introduction method of an initialization factor but also by a subsequent culture condition at the time of initialization induction.

iPS cells established in the above manner are subdivided and stored (for example, preservation by freezing) into storage containers such as vials after expansion culture for furnishing or sales thereof as iPS cell stocks to organizations using iPS cells such as universities or companies; or they are provided for differentiation induction after expansion culture for production of formulations to be administered patients. Methods for culturing iPS cells in these expansion cultures are roughly divided into adherent culture where cells are adhered to and cultured on a flat substrate, and suspension culture where cells are suspended and cultured in a liquid medium. The number of cells on a surface of the substrate obtained by adherent culture depends on an area for culture; and therefore, a huge area is required for scale-up and this makes it difficult to supply an amount of cells needed for regenerative medicine. In contrast, suspension culture allows cells to be cultured while being suspended in a liquid medium and this easily enables scale-up, and this is suitable for mass production of cells. For example, Non Patent Literature 3 discloses a method of suspension-culturing pluripotent stem cells while liquid medium is stirred with use of a spinner flask as a cell culture vessel for suspension culture. In addition, Patent Literature 3 discloses a method for allowing a protein kinase Cβ (PKCβ) inhibitor and a tankyrase inhibitor (TNKS inhibitor) to coexist in a medium for maintenance of an undifferentiated state when pluripotent stem cells are cultured in suspension.

### CITATION LIST

### Patent Literature

Patent Literature 1: International Publication No. WO2007/069666
Patent Literature 2: Japanese Patent Laid-Open No. 2008-283972
Patent Literature 3: International Publication No. WO2021/162090 Non Patent Literature

### Non Patent Literature

Non Patent Literature 1: Takahashi K. and Yamanaka S., Cell, 2006, August 25; 126(4):663-76.
Non Patent Literature 2: Takahashi K. et al., Cell, 2007, November 30; 131(5):861-72.
Non Patent Literature 3: Sumi T, et al., PLoS One, 2013; 8(5):e63378

### SUMMARY OF INVENTION

### Technical Problem

As described above, a method for performing suspension culture of iPS cells that have completed their establishment while maintaining an undifferentiated state has been known. However, a method for performing a stage for initializing somatic cells and obtaining iPS cells is performed under a suspension culture condition and establishing high-purity iPS cells has not been known. Usually, establishment of iPS cells is carried out under an adherent culture condition where cells that are changing to iPS cells or iPS cells are likely to be stabilized; however, this requires use of feeder cells or extracellular matrices, and it is disadvantageous that high-quality iPS cells are not always obtained depending on the difference in the type, culture condition or the like of feeder cells or extracellular matrices to be used. Further, the inventors have actually established iPS cells under the suspension condition in a general-purpose medium without use of feeder cells or extracellular matrices and they have found that the purity of obtained iPS cells was low and the efficiency of establishment was bad.

An object of the present invention is to provide a method for producing iPS cells, which enables initialization culture of somatic cells by a suspension method such that iPS cells to be established stably have high purity without using feeder cells and adhering cells to a substrate.

### Solution to Problem

The present invention includes the following.

(1) A method for producing an induced pluripotent stem cell (iPS cell), comprising the following steps I and II:
I. introducing an initialization gene into a somatic cell; and
II. performing initialization and amplification culture of the cell into which the gene has been introduced, in a liquid medium comprising at least one selected from the group consisting of a protein kinase Cβ (PKCβ) inhibitor and a WNT inhibitor under suspension culture condition.

(2) The method according to (1), further comprising the following steps III and IV:
III. sorting an undifferentiation marker positive cell from the cell obtained in the step II; and
IV. performing suspension culture of the cell sorted in the step III to obtain a cell population.

(3) The method according to (2), wherein the step III is a step of fractionating the sorted cell.

(4) The method according to (2) or (3), wherein the undifferentiation marker is at least one selected from the group consisting of SSEA-4, TRA-1-60, TRA-1-81 and TRA-2-49.

(5) The method according to any one of (1) to (4), wherein the somatic cell is a blood mononuclear cell.

(6) The method according to any one of (1) to (5), wherein the gene to be introduced into the somatic cell is incorporated into an episomal plasmid or sendai virus and introduced into the somatic cell.

(7) The method according to any one of (1) to (6), wherein the initialization gene to be introduced comprises at least one gene selected from the group consisting of an OCT3/4 gene, a KLF4 gene and an L-MYC gene.

(8) The method according to any one of (1) to (7), wherein the liquid medium used in and after the step II comprises at least one selected from L-ascorbic acid, insulin, transferrin, selenium and sodium hydrogen carbonate.

(9) The method according to any one of (1) to (8), wherein the liquid medium used in and after the step II comprises FGF2 and/or TGF-β1.

(10) The method according to any one of (1) to (9), wherein the liquid medium used in and after the step II comprises a ROCK inhibitor.

(11) The method according to (10), wherein the ROCK inhibitor is Y-27632.

(12) The method according to any one of (2) to (11), wherein, in the cell population obtained in the step IV, the proportion of cells positive for OCT4 is 90% or higher, and the proportion of cells positive for NANOG is 90% or higher.

(13) The method according to any one of (1) to (12), wherein the step II comprises a procedure of colleting a cell aggregate.

(14) The method according to any one of (2) to (13), wherein the step IV comprises a procedure of collecting a cell aggregate.

(15) A cell population comprising iPS cells produced by the method according to any one of (1) to (14).

(16) A medium for initialization and amplification culture of a somatic cell, wherein the medium is a liquid medium comprising at least one selected from the group consisting of a PKCβ inhibitor and a WNT inhibitor.

(17) The medium according to (16), wherein the medium comprises at least one selected from the group consisting of L-ascorbic acid, insulin, transferrin, selenium and sodium hydrogen carbonate.

(18) The medium according to (16) or (17), wherein the medium comprises FGF2 and/or TGF-β1.

(19) The medium according to any one of (16) to (18), wherein the medium comprises a ROCK inhibitor.

(20) A composition for initialization and amplification culture of a somatic cell, wherein the composition comprises at least one selected from the group consisting of a PKCβ inhibitor and a WNT inhibitor for addition to a medium for initialization and amplification culture of a somatic cell.

(21) The composition according to (20), wherein the composition comprises at least one selected from the group consisting of L-ascorbic acid, insulin, transferrin, selenium and sodium hydrogen carbonate.

(22) The composition according to (20) or (21), wherein the composition comprises FGF2 and/or TGF-β1.

(23) The composition according to any one of (20) to (22), wherein the composition comprises a ROCK inhibitor.

(24) The method according to any one of (1) to (14), wherein the step II is a step of performing initialization and amplification culture of the cell into which the gene has been introduced, in a non-gelled medium that is in a fluid state under a suspension culture condition.

(25) The method according to (24), wherein the amplification culture in the step II is performed by a suspension rotational method and a rotational speed thereof is 10 to 100 rpm.

(26) The method according to (24), wherein the amplification culture in the step II is performed by a suspension stirring method and for a rotational speed of a stirring blade thereof, a blade tip speed is in the range of 0.05 to 1.37 m/s.

(27) The method according to (26), wherein the amplification culture comprises a subculture accompanied by a culture scale modification and a rotational frequency of the stirring blade is determined such that a change in the power required for stirring per volume or the blade tip speed is less than 10% before and after the culture scale modification.

The present specification includes the contents disclosed in Japanese Patent Application No. 2022-016513, on the basis of which the priority of the present application is claimed.

### Advantageous Effects of Invention

The present invention can provide a method for producing iPS cells, which enables initialization culture of somatic cells by a suspension method such that iPS cells to be established stably have high purity without using feeder cells and adhering cells to a substrate.

### BRIEF DESCRIPTION OF DRAWINGS

FIG. 1 shows results of flow cytometry of cells prepared by suspension culture of cells initialized using an episomal plasmid in the presence of PKCβ and WNT inhibitors, wherein the flow cytometry is performed using TRA-1-60 and SSEA-4 antibodies. (A) shows a dot plot of side scattering light (BSC) against forward scattering light (FSC) in all events. Gating on a portion encircled by a line in the figure is performed. (B) show a dot plot of fluorescence intensities of TRA-1-60 and fluorescence intensities of SSEA-4 of cells that are subjected to gating in (A). Cells in the range encircled by the line in the figure are sorted. (C) shows a histogram plot of fluorescence intensities of TRA-1-60 and (D) shows a histogram plot of fluorescence intensities of SSEA-4.

FIG. 2 shows results of flow cytometry of cells prepared by suspension culture of cells initialized using an episomal plasmid in the absence of PKCβ and WNT inhibitors, wherein the flow cytometry is performed using TRA-1-60 and SSEA-4 antibodies. (A) shows a dot plot of side scattering light (BSC) against forward scattering light (FSC) in all events. Gating on a portion encircled by a line in the figure is performed. (B) show a dot plot of fluorescence intensities of TRA-1-60 and fluorescence intensities of SSEA-4 of cells that are subjected to gating in (A). Cells in the range encircled by the line in the figure are sorted. (C) shows a histogram plot of fluorescence intensities of TRA-1-60 and (D) shows a histogram plot of fluorescence intensities of SSEA-4.

FIG. 3 shows microphotographs of cell aggregates in the 5th subculture prepared by suspension culture of cells that are initialized using an episomal plasmid and sorted by flow cytometry in Example 2 in the presence and absence of PKCβ and WNT inhibitors. (A) is a microphotograph of cell aggregates cultured in the presence of PKCβ and WNT inhibitors, and (B) is a microphotograph of cell aggregates cultured in the absence of PKCβ and WNT inhibitors.

FIG. 4 shows microphotographs of cell aggregates in the 11th subculture prepared by suspension culture of 3 clones (#1, #2 and #3) of initialized cells in the presence of PKCβ and WNT inhibitors. (A), (B) and (C) are microphotographs of cell aggregates for clones #1, #2 and #3, respectively.

### DESCRIPTION OF EMBODIMENTS

### 1. Method for Producing Induced Pluripotent Stem Cells (iPS Cells)

### 1-1. Summary

A method for producing iPS cells according to the present invention (hereinafter, also referred to as “the method of the present invention”) is characterized by comprising the following steps I and II:
I. introducing an initialization gene into a somatic cell; and
II. performing initialization and amplification culture of the cell into which the gene has been introduced, in a liquid medium comprising at least one selected from the group consisting of a protein kinase Cβ (PKCβ) inhibitor and a WNT inhibitor under a suspension culture condition.

Further, the method of the present invention optionally comprises the following steps III and IV:
III. sorting an undifferentiation marker positive cell from the cell obtained in the step II; and
IV. performing suspension culture of the cell sorted in the step III to obtain a cell population.

The method of the present invention has the above features and thereby, it can provide iPS cells without using feeder cells and adhering cells to a substrate under a suspension culture condition.

### 1-2. Definition of Terms

The terms to be used in the present specification will be defined below.

### <Cells>

A “pluripotent stem cell” in the present specification refers to a cell having pluripotent capacity (pluripotency) to differentiate into almost all types of cells (tissue cells or germ cells) constituting a living body and being capable of permanently continuing proliferation with the pluripotency maintained in in vitro culture under proper conditions. More specifically, pluripotency means an ability to differentiate into germ layers constituting an individual (for vertebrates, three germ layers: ectoderm, mesoderm, and endoderm). Examples of such cells include embryonic stem cells (ES cells), embryonic germ cells (EG cells), germline stem cells (GS cells), and induced pluripotent stem cells (iPS cells). An “iPS cell” is a subject matter of the invention in the present specification and refers to a pluripotent stem cell that has been initialized by introducing a few genes encoding initialization factors into a differentiated somatic cell to turn the somatic cell into an undifferentiated state.

A “somatic cell” in the present specification refers to cells other than a germ cell among cells constituting an animal living body. A somatic cell in the present specification is not limited as long as it is a cell that can obtain the pluripotency by initialization induction. Further, the biological species, from which a somatic cell is derived, are not particularly limited as long as they are multicellular organisms; however, the somatic cell is preferably an animal-derived cell, in particular a mammalian-derived cell. Examples of the cells include cells derived from a rodent such as a mouse, a rat, a hamster, and a guinea pig, a domestic or pet animal such as a dog, a cat, a rabbit, a bovine, a horse, sheep and a goat, and a primate such as a human, a rhesus monkey, a gorilla, and a chimpanzee. Particularly preferred are cells derived from a human. A tissue or organ, from which a somatic cell is derived, is not particularly limited, but preferred is a tissue or organ from which a somatic cell is easily collected and in which initialization is efficiently induced. The tissue or organ may be, for example, skin, an organ such as a liver, blood, urine, a cancer tissue, a dental pulp cell or the like. Further, whether a somatic cell is a differentiated cell or undifferentiated cell is not an issue; and it may be an established cell line or a primary cultured cell isolated from a tissue. Preferably, it is a differentiated cell. Examples of the somatic cell in the present specification include a human fibroblast, a human epithelial cell, a human liver cells, a human blood cell, a mesenchymal cell, a nerve cell and a muscle cell. Among these, a suspension-culturable cell is preferred.

### <Initialization (Reprogramming)>

“Initialization (reprogramming)” in the present specification refers to an operation or process to change a somatic cell to another cell species. In general, it means to change into an undifferentiated cell by dedifferentiating a differentiated cell. In the present specification, it refers to an operation or process to change a somatic cell to an iPS cell unless otherwise stated.

“Initialization induction” or “induce initialization” in the present specification means that an operation that can cause initialization is given to a cell to actually turn the cell into an initialized state. In this connection, “perform initialization induction” means to give an operation that can cause initialization to a cell, and whether or not to be actually initialized is not an issue. For example, “perform initialization induction” is to perform an operation to introduce a reprogramming factor needed for initialization into a somatic cell and culture the factor-induced somatic cell under a predetermined condition.

“Initialization factor (reprogramming factor)” in the present specification refers to a factor that can cause initialization of a somatic cell by introducing into the somatic cell the factor alone or the factor with another factor. When “initialization factor” is simply referred to without specifying a protein or gene in the present specification, the initialization factor means any of the protein, a nucleic acid encoding the protein and a gene expression vector comprising the nucleic acid. In the present specification, in particular, the nucleic acid encoding the protein is also called “initialization gene.” Examples of the initialization factor include any of 4 factors of OCT3/4, SOX2, KLF4 and C-MYC (often referred to as “4 initialization factors” in the present specification), and factors associated with any of the 4 initialization factors.

In the present specification, “associated factors” with the 4 initialization factors refer to a factor that can induce initialization of a somatic cell when it is introduced into the somatic cell instead of any of the 4 initialization factors.

In the present specification, the animal species from which an initialization factor is derived is not an issue. The animal species from which an initialization factor is derived is, for example, mammalian species. The mammalian species may be, for example, any of a human, a mouse, a rat, a hamster, a guinea pig, a dog, a cat, a rabbit, a bovine, a horse, sheep, a goat, a rhesus monkey, a gorilla, and a chimpanzee. A human is preferred.

Examples of the initialization factors and associated factors thereof are shown below, but the initialization factors and associated factors thereof in the present specification are not limited to the following examples.

Specific examples of OCT3/4 include a human OCT3/4 protein consisting of an amino acid sequence as set forth in SEQ ID NO:1. Examples of the associated factors of OCT3/4 include NR5A2 (LRH1) and TBX3.

Specific examples of KLF4 include a human KLF4 protein consisting of an amino acid sequence as set forth in SEQ ID NO:4. Examples of the associated factors of KLF4 include KLF1, KLF2, KLF5 and mutant KLF of the present invention, and examples thereof include a human KLF1 protein consisting of an amino acid sequence as set forth in SEQ ID NO:2, a human KLF2 protein consisting of an amino acid sequence as set forth in SEQ ID NO:3, and a human KLF5 protein consisting of an amino acid sequence as set forth in SEQ ID NO:5.

Specific examples of C-MYC include a human C-MYC protein consisting of an amino acid sequence as set forth in SEQ ID NO:6. Examples of the associated factors of C-MYC include T58A mutant of C-MYC, N-MYC and L-MYC. Examples thereof include a human N-MYC protein consisting of an amino acid sequence as set forth in SEQ ID NO:7 and a human L-MYC protein consisting of an amino acid sequence as set forth in SEQ ID NO:8.

Specific examples of SOX2 include a human SOX2 protein consisting of an amino acid sequence as set forth in SEQ ID NO:10. Examples of the associated factors of SOX2 include SOX1, SOX3, SOX15 and SOX18. Examples thereof include a human SOX1 protein consisting of an amino acid sequence as set forth in SEQ ID NO:9, a human SOX3 protein consisting of an amino acid sequence as set forth in SEQ ID NO:11, a human SOX15 protein consisting of an amino acid sequence as set forth in SEQ ID NO:12, and a human SOX18 protein consisting of an amino acid sequence as set forth in SEQ ID NO:13.

In addition to the above, examples of initialization factors and associated factors thereof include LIN28A, LIN28B, LIN41, GLIS1, FOXH1 and HMGA2.

Note that, in the present specification, a cell obtained by performing the above-described initialization induction on a somatic cell is also called “initialized cell.” The initialized cell is preferably a cell obtained by introducing the above initialization factor (reprogramming factor) into a somatic cell, and further preferably a cell obtained by introducing the initialization gene into a somatic cell. In general, an iPS-like cell when and after initialization induction is performed on a somatic cell, or a cell that will become an iPS cell is sometimes referred to as “initialized cell.” However, in the present specification, cells that are the subject during a period in which initialization and amplification culture are performed on a somatic cell since initialization induction is performed, that is a period of the step II, are collectively referred to and defined as “initialized cell” and therefore, part of iPS-like cells or iPS cells may be included therein.

### <Cell Aggregate>

In the present specification, a “cell aggregate” is a massive cell population formed through cell aggregation in suspension culture, and is also called a spheroid or aggregate. Cell aggregates are generally spherical in typical cases. Cells constituting a cell aggregate are not limited as long as they are one or more types of the aforementioned cells. For example, a cell aggregate composed of human iPS cells include cells expressing a undifferentiated marker and/or being positive for a undifferentiated marker.

Undifferentiated markers are gene markers specifically or excessively expressed in iPS cells, and examples thereof can include Alkaline Phosphatase, NANOG, OCT4, SOX2, TRA-1-60, TRA-1-81, TRA-2-49, c-Myc, KLF4, LIN28, SSEA-4, and SSEA-1.

Undifferentiated markers can be detected with any detection method in the art. Examples of methods for detecting cell markers include, but are not limited, flow cytometry. In the case that a fluorescence-labeled antibody is used as a detection reagent in flow cytometry, if a cell emitting more intense fluorescence than a negative control (isotype control) is detected, the cell is determined as “positive” for the marker. The proportion of cells positive for a fluorescence-labeled antibody as analyzed by flow cytometry is occasionally referred to as the “positive rate”. Any antibody known in the art can be used for such fluorescence-labeled antibodies, and examples thereof include, but are not limited to, antibodies labeled with fluorescein isothiocyanate (FITC), phycoerythrin (PE), or allophycocyanin (APC).

If cells constituting a cell aggregate are iPS cells, the positive rate for a undifferentiated marker can be preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably over 99% and 100% or lower. Cell aggregates in which the percentage of cells expressing an undifferentiated marker and/or being positive for an undifferentiated marker falls within the range are highly undifferentiated, highly homogeneous cell populations.

### <Culture and Medium>

Cell culture methods are roughly classified into “suspension culture method” and “adherent culture method.” In the present specification, a “suspension culture method” is a method of suspension-culturing cells, and cells in this method are present usually as a cell cluster formed through aggregation on and after the middle stage of culture though cells are present as a single cell in a culture solution in the early stage of culture. “Suspension culture” refers to allowing cells in a suspended state in medium to proliferate. The “suspended state” in the present specification refers to a state in which cells are not adhering to an external matrix such as a culture vessel. The “adherent culture method” is a method of adherent-culturing cells. “Adherent culture” refers to allowing cells to proliferate as a monolayer in principle with the cells adhered to an external matrix or the like such as a culture vessel. In the present specification, a culture wherein microcarriers having cells adhered thereto are suspended is regarded as adherent culture since cells themselves are adhered to external matrices. Note that the above-described iPS cell and initialized cell can be usually cultured not only by adherent culture but also by suspension culture.

In the present invention, “medium” refers to a liquid or solid substance prepared for culturing cells. In principle, medium comprises components indispensable for proliferation and/or maintenance of cells over their minimum requirements. Unless otherwise stated, liquid medium for animal cells for use in culture of cells derived from an animal is employed as medium in the present specification.

In the present specification, “minimal essential medium” refers to medium that serves as a base for media for various animal cells. Even minimal essential medium alone is applicable to culture, and media specific to different cells for different purposes may be prepared by adding various culture additives. Examples of minimal essential medium applicable in the present specification include, but are not limited to, BME medium, BGJb medium, CMRL1066 medium, Glasgow MEM medium, Improved MEM Zinc Option medium, IMDM medium (Iscove'S Modified Dulbecco'S Medium), Medium 199 medium, Eagle MEM medium, αMEM medium, DMEM medium (Dulbecco'S Modified Eagle'S Medium), Ham's F10 medium, Ham's F12 medium, RPMI 1640 medium, Fischer's medium, and mixed media of them (e.g., DMEM/F12 medium (Dulbecco'S Modified Eagle'S Medium/Nutrient Mixture F-12 Ham)). For DMEM/F12 medium, in particular, medium obtained by mixing DMEM medium and Ham's F12 medium at a weight ratio preferably in the range of 60/40 or higher and 40/60 or lower, such as 58/42, 55/45, 52/48, 50/50, 48/52, 45/55, and 42/58, is used. In addition, other media used for culture of human iPS cells and human ES cells can be preferably used. Medium to be used in the present invention is preferably medium comprising no serum, in other words, serum-free medium.

In the present specification, a “culture additive” is a substance that is added to medium for culture, except serum. Specific examples of culture additives include, but are not limited to, L-ascorbic acid, insulin, transferrin, selenium, sodium hydrogen carbonate, growth factors, fatty acid or lipid, amino acids (e.g., non-essential amino acids), vitamins, cytokine, antioxidants, 2-mercaptoethanol, pyruvic acid, buffering agents, inorganic salts, and antibiotics. The insulin, transferrin, and cytokine may be naturally occurring ones separated from tissue or serum or the like of an animal (preferably, human, mouse, rat, bovine, horse, goat, etc.), or recombinant proteins produced with a gene engineering technique. Examples of applicable growth factors include, but are not limited to, FGF2 (Basic fibroblast growth factor-2), TGF-β1 (Transforming growth factor-§ 1), Activin A, IGF-1, MCP-1, IL-6, PAI, PEDF, IGFBP-2, LIF, and IGFBP-7. Examples of applicable antibiotics include, but are not limited to, penicillin, streptomycin, and amphotericin B. FGF2 and/or TGF-β1 is a particularly preferable growth factor as a culture additive for medium to be used in the present invention.

It is preferable that each medium comprise a ROCK inhibitor. In particular, when cells are in a single cell state, it is preferable to comprise a ROCK inhibitor. Examples of ROCK inhibitors include Y-27632. Cell death in suspension culture of iPS cells can be significantly reduced by inclusion of a ROCK inhibitor in medium.

Medium to be used in the present invention can comprise one or more of the culture additives. Medium to which the culture additives are to be added is typically any of the minimal essential media, but is not limited thereto.

The culture additives in the form of a solution, derivatives, salts, or a mixed reagent or the like can be added to medium. For example, L-ascorbic acid in the form of a derivative such as magnesium ascorbyl-2-phosphate may be added to medium, and selenium in the form of a selenite (such as sodium selenite) may be added to medium. Insulin, transferrin, and selenium in the form of an ITS reagent (insulin-transferrin-selenium) can be added to medium.

Commercially available medium to which at least one selected from L-ascorbic acid, insulin, transferrin, selenium, and sodium hydrogen carbonate may be used. Examples of commercially available medium to which insulin and transferrin have been added include CHO—S-SFM II (Life Technologies Japan Ltd.), Hybridoma-SFM (Life Technologies Japan Ltd.), eRDF Dry Powdered Media (Life Technologies Japan Ltd.), UltraCULTURE™ (BioWhittaker), UltraDOMA™ (BioWhittaker), UltraCHO™ (BioWhittaker), UltraMDCKT™ (BioWhittaker), STEMPRO® hESC SFM (Life Technologies Japan Ltd.), Essential8™ (Life Technologies Japan Ltd.), StemFit® AK02N (Ajinomoto Co., Inc.), mTeSR1 (VERITAS Corporation), and TeSR2 (VERITAS Corporation).

A preferable medium to be used in the present invention is a liquid medium comprising at least one selected from the group consisting of L-ascorbic acid, insulin, transferrin, selenium, and sodium hydrogen carbonate. Further, a preferable medium to be used in the present invention is a liquid medium comprising at least one growth factor (preferably FGF2 and/or TGF-β1). Particularly preferred is DMEM/F12 medium comprising L-ascorbic acid, insulin, transferrin, selenium, and sodium hydrogen carbonate, and at least one growth factor (preferably FGF2 and/or TGF-β1), and being free of serum.

### <PKCβ Inhibitor>

In the present specification, the term “protein kinase Cβ (PKCβ) inhibitor” means a substance that inhibits or suppresses the activity of PKCβ. Protein kinases each have a catalytic region in the C-terminal side and a regulatory region in the N-terminal side. The catalytic region is composed of a sequence that recognizes phosphorylated residues on a substrate protein and a sequence that forms an ATP/Mg²⁺ bond. The regulatory region is composed of C1 and C2 domains.

PKC includes PKCα, PKCβI, PKCβII, and PKCy as conventional isozymes. PKC includes PKCδ, PKCε, PKCθ, and PKCη as novel isozymes, and PKCζ, PKCλ, and PKCμ as atypical isozymes.

In the present specification, the term PKCβ means both of those PKCβI and PKCβII, or one of PKCβI and PKCβII. In the present specification, the term PKCβ inhibitor means a substance that inhibits at least PKCβI and/or PKCβII among those conventional, novel, and atypical isozymes. That is, the term PKCβ inhibitor means any of a substance that inhibits or suppresses only the activity of PKCβI, a substance that inhibits or suppresses only the activity of PKCβII, and a substance that inhibits or suppresses the activities of PKCβI and PKCβII.

The PKCβ inhibitor may be a substance that specifically inhibits or suppresses only the activity of PKCβ, and may be a substance that inhibits or suppresses the activity of another isozyme in addition to that of PKCβI or PKCβII. For example, the PKCβ inhibitor may be a substance that inhibits or suppresses the activities of all the aforementioned conventional, novel, and atypical isozymes including PKCβI and PKCβII. The PKCβ inhibitor may be a substance that inhibits or suppresses the activities of the conventional isozymes PKCα and PKCγ in addition to those of PKCβI and PKCβII. Moreover, the PKCβ inhibitor may be a substance that inhibits or suppresses the activities of the novel isozymes PKCδ, PKCε, PKCθ, and PKCη in addition to those of PKCβI and PKCβII.

Examples of the PKCβ inhibitor include a compound that directly or indirectly acts on PKCβ, an antisense nucleic acid for a gene encoding PKCβ, an RNA-interference-inducible nucleic acid (e.g., siRNA), a dominant-negative mutant, and an expression vector for any of them.

An example of the PKCβ inhibitor can be a compound having the following structural formula [Formula I].

A compound represented by:

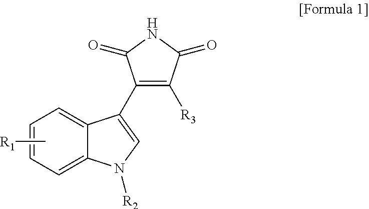

or a salt thereof.

In Formula I,
R₁ is a hydrogen atom or an alkoxy group having 1 to 3 carbon atoms (preferably a methoxy group), R₂ is a hydrogen atom, an alkyl group having 1 to 3 carbon atoms (preferably a methyl group), or an alkyl group having 1 to 3 carbon atoms and substituted with —N(R_{A})₂ (preferably —(CH₂)₃—N(CH₃)₂),
R_{A} is independently an ethyl group or a methyl group (preferably a methyl group),
R₃ is a group represented by

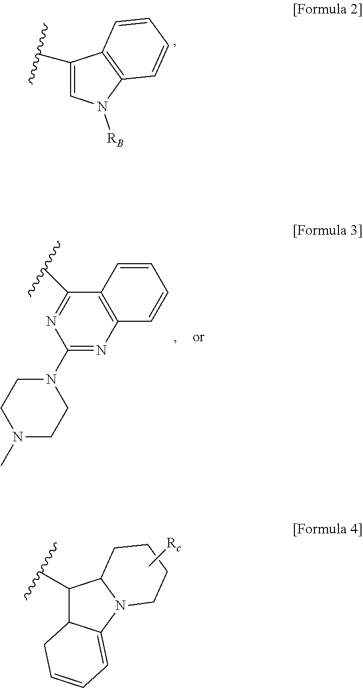

R_{B} is a hydrogen atom, an alkyl group having 2 to 4 carbon atoms and substituted with —S—C(═NH)(—NH₂) (preferably —(CH₂)₃—S—C(═NH)(—NH₂)), or a group represented by

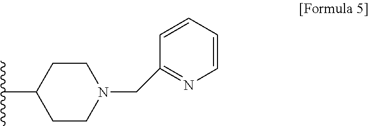 [SMILES: *C1CCN(Cc2ccccn2)CC1]

or R₂ and R_{B} are optionally integrated together to form the following divalent group:

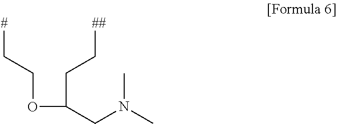

wherein # indicates bonding to the bonding position of R₂, and ## indicates bonding to the bonding position of R_{B},
the configuration of the asymmetric carbon comprised in the divalent group is preferably, but is not limited to:

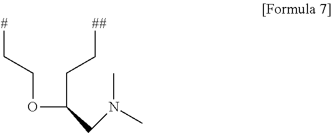

R_{C} is an alkyl group having 1 to 3 carbon atoms and substituted with —N(R_{D})₂ (preferably —(CH₂)—N(CH₃)₂), and
R_{D} is independently an ethyl group or a methyl group (preferably a methyl group).

Examples of the salt of the compound represented by Formula I can include a hydrochloride and a sulfate.

Specific examples of the PKCβ inhibitor having the above structural formula [Formula I] include a compound selected from the group consisting of Go6983, GF109203X, LY-333531, Enzastaurin, Sotrastaurin, Ro-31-8220-mesylate, Ro-32-0432-hydrochloride, Go6976, Rottlerin, Midostaurin, Daphnetin, Dequalinium Chloride, Baicalein, Quercetin, Luteolin, Bisindolylmaleimide II, Calphostin C, Chelerythrine chloride, L-threo Dihydrosphingosine, and Melittin. Among the PKCβ inhibitors having the above structural formula, a compound selected from the group consisting of Go6983, GF109203X, and LY-333531 is preferably used.

The structural formula of Go6983 (3-[1-[3-(dimethylamino)propyl]-5-methoxy-1H-indol-3-yl]-4-(1H-indol-3-yl)-1H-pyrrole-2,5-dione) is shown in the following.

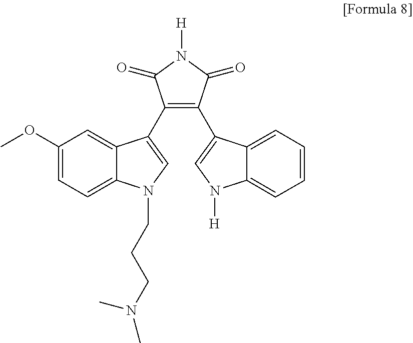 [SMILES: COc1ccc2c(c1)c(C1=C(c3c[nH]c4ccccc34)C(=O)NC1=O)cn2CCCN(C)C]

The structural formula of GF109203X (2-[1-(3-dimethylaminopropyl)indol-3-yl]-3-(indol-3-yl)maleimide) is shown in the following.

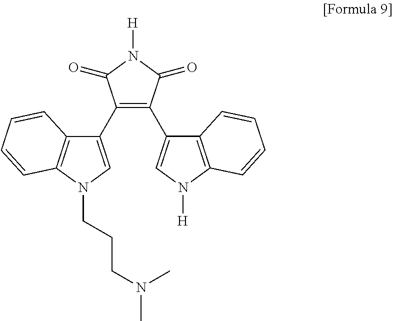 [SMILES: CN(C)CCCn1cc(C2=C(c3c[nH]c4ccccc34)C(=O)NC2=O)c2ccccc21]

The structural formula of LY-333531 ((9S)-[(dimethylamino)methyl]-6,7,10,11-tetrahydro-9H,18H-5,21:12,17-dimethenodibenzo[e,k]pyrrolo[3,4-h][1,4,13]oxsadiazacyclohexadecine-18,20(19H)-dione, monohydroxychloride) is shown in the following.

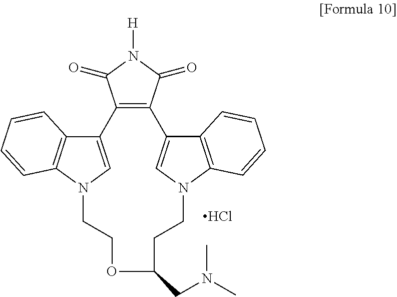 [SMILES: CN(C)C[C@@H]1CCn2cc(c3ccccc32)C2=C(C(=O)NC2=O)c2cn(c3ccccc23)CCO1 ; Cl]

The structural formula of Enzastaurin (3-(1-methylindol-3-yl)-4-[1-[1-(pyridin-2-ylmethyl)piperidin-4-yl]indol-3-yl]pyrrole-2,5-dione) is shown in the following.

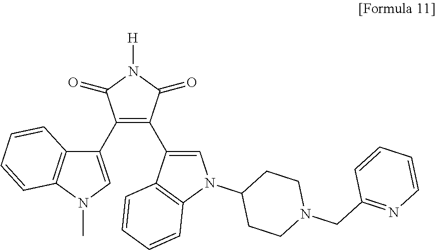 [SMILES: Cn1cc(C2=C(c3cn(C4CCN(Cc5ccccn5)CC4)c4ccccc34)C(=O)NC2=O)c2ccccc21]

The structural formula of Sotrastaurin (3-(1H-indol-3-yl)-4-(2-(4-methylpiperazin-1-yl)quinazolin-4-yl)-1H-pyrrole-2,5-dione) is shown in the following.

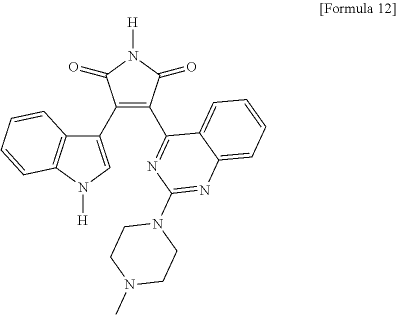 [SMILES: CN1CCN(c2nc(C3=C(c4c[nH]c5ccccc45)C(=O)NC3=O)c3ccccc3n2)CC1]

The structural formula of Ro-31-8220-mesylate (3-[3-[2,5-dihydro-4-(1-methyl-1H-indol-3-yl)-2,5-dioxo-1H-pyrrole-3-yl]-1H-indol-1-yl]propyl carbamimidothioate mesylate) is shown in the following.

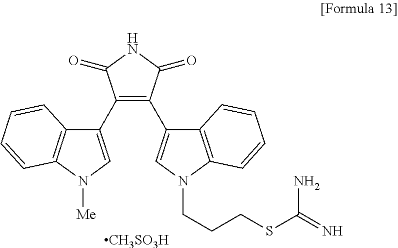

The structural formula of Ro-32-0432-hydrochloride (3-[(8S)-8-[(dimethylamino)methyl]-6,7,8,9-tetrahydropyrido[1,2-a]indol-10-yl]-4-(1-methyl-1H-indol-3-yl)-1H-pyrrole-2,5-dione hydrochloride) is shown in the following.

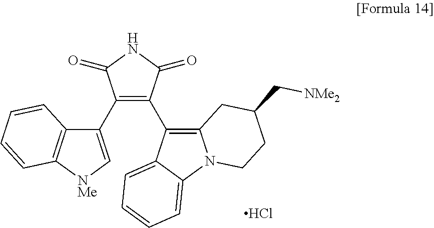 [SMILES: CN(C)C[C@H]1CCn2c(c(C3=C(c4cn(C)c5ccccc45)C(=O)NC3=O)c3ccccc32)C1 ; Cl]

### <Wnt Inhibitor>

“WNT inhibitor” used herein refers to entire of substances that inhibit a transduction pathway of WNT signaling proteins (WNT signaling transduction pathway). It is not particularly limited as long as it is a substance that inhibits a WNT signaling transduction pathway; and examples of representative WNT inhibitors include a tankyrase (TNKS) inhibitor and Porcupine (PORCN) inhibitor.

In the present specification, the term “tankyrase (TNKS) inhibitor” means a substance that inhibits or suppresses the activity of tankyrase. Tankyrase belongs to the poly(ADP-ribose) polymerase (PARP) family to poly(ADP-ribosylate) a target protein, and tankyrase 1 (tankyrase-1/PARP-5a) and tankyrase 2 (tankyrase-2/PARP-5b) are known. Tankyrase is known to have a function to promote the telomere elongation by telomerase through poly(ADP-ribosylation) of the telomere protein TRF1 to liberate it from a telomere.

In the present specification, the term TNKS means both of those tankyrase 1 and tankyrase 2, or one of tankyrase 1 and tankyrase 2. In the present specification, the term TNKS inhibitor means a substance that inhibits tankyrase 1 and/or tankyrase 2. That is, the term TNKS inhibitor means any of a substance that inhibits or suppresses only the activity of tankyrase 1, a substance that inhibits or suppresses only the activity of tankyrase 2, and a substance that inhibits or suppresses the activities of tankyrase 1 and tankyrase 2.

Examples of the TNKS inhibitor include a compound that directly or indirectly acts on TNKS, an antisense nucleic acid for a gene encoding TNKS, an RNA-interference-inducible nucleic acid (e.g., siRNA), a dominant-negative mutant, and an expression vector for any of them.

An example of the TNKS inhibitor can be a compound selected from the group consisting of IWR-1-endo, XAV939, G007-LK, G244-LM, MSC2504877, and WIKI4. Among the TNKS inhibitors, IWR-1-endo and/or XAV939 is particularly preferably used.

The structural formula of IWR-1-endo (4-(1,3,3a,4,7,7a-hexahydro-1,3-dioxo-4,7-methano-2H-isoindol-2-yl)-N-8-quinolinyl-benzamide) is shown in the following.

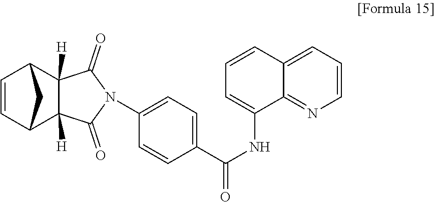 [SMILES: O=C(Nc1cccc2cccnc12)c1ccc(N2C(=O)[C@@H]3[C@H](C2=O)[C@@H]2C=C[C@H]3C2)cc1]

The structural formula of XAV939 (3,5,7,8-tetrahydro-2-[4-(trifluoromethyl)phenyl]-4H-thiopyrano[4,3-d]pyrimidin-4-one) is shown in the following.

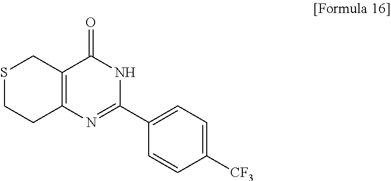 [SMILES: O=c1[nH]c(-c2ccc(C(F)(F)F)cc2)nc2c1CSCC2]

The structural formula of G007-LK (4-[5-[(IE)-2-[4-(2-chlorophenyl)-5-[5-(methylsulfonyl)-2-pyridinyl]-4H-1,2,4-thiazol-3-yl]ethenyl]-1,3,4-oxsadiazol-2-yl]-benzonitrile) is shown in the following.

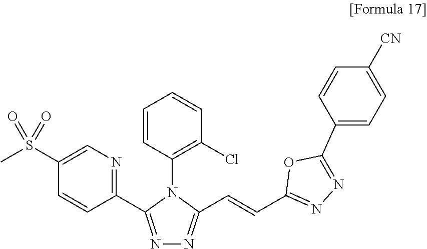 [SMILES: CS(=O)(=O)c1ccc(-c2nnc(/C=C/c3nnc(-c4ccc(C#N)cc4)o3)n2-c2ccccc2Cl)nc1]

The structural formula of G244-LM (3,5,7,8-tetrahydro-2-[4-[2-(methylsulfonyl)phenyl]-1-piperazinyl]-4H-thiopyrano[4,3-d]pyrimidin-4-one) is shown in the following.

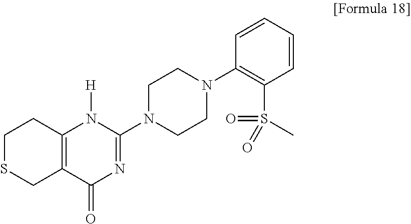 [SMILES: CS(=O)(=O)c1ccccc1N1CCN(c2nc(=O)c3c([nH]2)CCSC3)CC1]

The structural formula of WIKI4 (2-[3-[[4-(4-methoxyphenyl)-5-(4-pyridinyl)-4H-1,2,4-thiazol-3-yl]thio]propyl]-1H-benzo[de]isoquinoline-1,3(2H)-dione) is shown in the following.

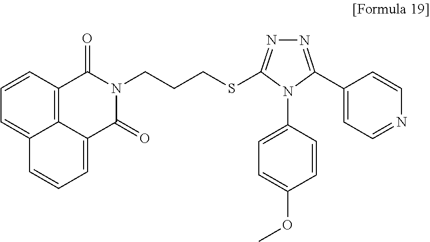 [SMILES: COc1ccc(-n2c(SCCCN3C(=O)c4cccc5cccc(c45)C3=O)nnc2-c2ccncc2)cc1]

“Porcupine (PORCN) inhibitor” used herein refers to a substance that inhibits or suppresses the activity of Porcupine. Porcupine is an enzyme that catalyzes the addition of palmitoleic acid to serine residues of WNT proteins, a process required for the secretion of WNT proteins. As the Porcupine (PORCN) inhibitor, IWP-2, WNT-C59 and others are known.

The structural formula of IWP-2 (N-(6-methyl-2-benzothiazolyl)-2-[(3,4,6,7-tetrahydro-4-oxy-3-phenylthieno[3,2-d]pyrimidin-2-yl)thio]-acetamide) is shown below.

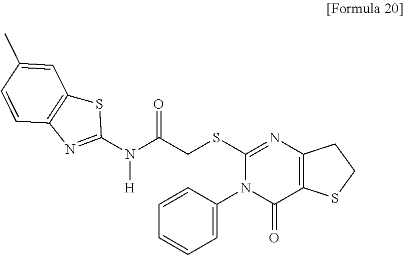 [SMILES: Cc1ccc2nc(NC(=O)CSc3nc4c(c(=O)n3-c3ccccc3)SCC4)sc2c1]

The structural formula of WNT-C59 (4-(2-methyl-4-pyridinyl)-N-[4-(3-pyridinyl)phenyl]benzene acetamide) is shown below.

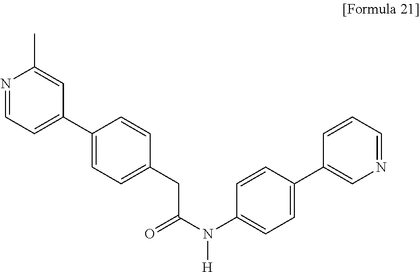 [SMILES: Cc1cc(-c2ccc(CC(=O)Nc3ccc(-c4cccnc4)cc3)cc2)ccn1]

### <Others>

“Plural” used herein refers to an integer such as 2 to 50, 2 to 45, 2 to 40, 2 to 35, 2 to 30, 2 to 25, 2 to 20, 2 to 15, 2 to 10, 2 to 7, 2 to 5, 2 to 4 or 2 to 3.

### 1-3. Configuration

As described above, the method of the present invention comprises, as essential elements, the following steps I and II:
I. introducing an initialization gene into a somatic cell, and
II. performing initialization and amplification culture of the cell into which the gene has been introduced, in a liquid medium comprising at least one selected from the group consisting of a protein kinase Cβ (PKCβ) inhibitor and a WNT inhibitor under suspension culture condition.

Further, the method of the present invention comprises, as optional elements, the following steps Ill and IV:
III. sorting an undifferentiation marker positive cell from the cell obtained in the step II; and
IV performing suspension culture of the cell sorted in the step III to obtain a cell population.

Hereafter, the elements of the method of the present invention are explained for each step.

### 1-3-1. Step of Introducing Initialization Gene (Step I)

In the step I in the method of the present invention, usable is a gene expression vector as means for introducing an initialization gene into a somatic cell. The step I has a procedure of bringing a somatic cell into contact with a reagent comprising a gene expression vector with an initialization gene (hereinafter, also referred to as “initialization inducer”).

### (Cell)

As described in the section “1-2. Definition of terms,” “somatic cells” in the method of the present invention indicates cells other than germ cells among cells forming an animal individual; however, in particular, blood mononuclear cells, more specifically peripheral blood mononuclear cells can be suitably used from the viewpoint that they are relatively low invasive and easily collected, are relatively easily induced for initialization, and are suspension cells. Mononuclear cells are not particularly limited, but active T cells, for example, are suitably used. Cells used herein for introducing an initialization gene are required to be present in a liquid medium, or a special solution for carrying out gene transfer by electroporation or the like. As long as being in such solutions, cells may be suspended or adhered; however, cells are preferably suspended. These suspension cells may be cells cultured in suspension in a liquid medium, and they may be cells that are cultured in adhesion and peeled off from a substrate or the like, and then suspended.

### (Gene Expression Vector)

A gene expression vector used in the step I comprises a promoter and an initialization gene as essential elements. The “gene expression vector” used herein refers to a vector that comprises a gene or a gene fragment in an expressible state and includes an expression unit that can express that gene or the like. The “expressible state” used herein refers to a state wherein a gene to be expressed is placed at a downstream region of a promoter under the control of the promoter. The gene expression vector usable in the method of the present invention is a vector comprising an initialization factor in an expressible state and it can express an initialization factor or a peptide fragment thereof in a somatic cell.

Hereafter, gene expression vectors usable for the method of the present invention and promoters comprised in the vectors, and other optional components will be described. A vector usable as the gene expression vector is not particularly limited as long as it can express an initialization factor in a somatic cell. Examples thereof include a viral vector, a plasmid vector and an artificial chromosome vector.

In the method of the present invention, the viral vector usable as the gene expression vector is not particularly limited as long as it can infect a somatic cell as the subject of initialization and express an initialization factor in the somatic cell. Examples thereof include an adenovirus vector, an adeno-associated virus (AAV) vector, a retrovirus vector, a lentiviral vector, and a Sendai virus vector. Depending on the type of a viral vector, the size of DNA that can be carried on the vector, the type of a cell that the vector infects, the cytotoxicity, whether or not to be incorporated into a host genome, and the expression period are varied; and a viral vector can be appropriately selected in accordance with the type of a somatic cell as the subject of initialization. For example, a replication-defective and persistent Sendai virus vector (SeVdp vector) does not cause integration to a host genome and has a property to persistently stay in cytoplasm, and therefore, it is highly safe and in particular, it can be used suitably (Nishimura K., et al., J Biol Chem. 2011 Feb. 11; 286(6):4760-71.; Fusaki N., et al., Proc Jpn Acad Ser B Phys Biol Sci. 2009; 85(8):348-62).

In the method of the present invention, the plasmid vector usable as the gene expression vector is not particularly limited as long as it can express an initialization factor in a somatic cell when it is introduced into the somatic cell as the subject of initialization. The plasmid vector may be a replicable shuttle vector between a mammalian cell and a bacterium such as Escherichia coli. Specific examples of the plasmid vector include plasmids derived from E. coli (pBR322, pUC18, pUC19, pUC118, pUC119, pBluescript, etc.), plasmids derived from Actinomyces (pIJ486, etc.), plasmids derived from Bacillus subtilis (pUB110, pSH19, etc.), and plasmids derived from yeast (YEp13, YEp24, Ycp50, etc.), and in addition to these, commercially available vectors may be used. Specific examples of the commercially available vectors include CMV6-XL3 (OriGene), EGFP-C1, pGBT-9 (Clontech), pcDNA, pcDM8, and pREP4 (Thermo Fisher Scientific Inc.). In particular, an episomal plasmid that has oriP-EBNA1 incorporated thereinto, causes no integration to a host genome and is autonomously replicable can be suitably used. For example, usable is an iPS cell production kit, Human iPS Cell Generation™ Episomal Vector Mix (TaKaRa), which comprises an episomal plasmid having an initialization gene incorporated thereinto.

In the method of the present invention, examples of the artificial chromosome vector usable as the gene expression vector include a human artificial chromosome (HAC), a yeast artificial chromosome (YAC) and a bacterial artificial chromosome (BAC, PAC).

In the method of the present invention, the promoter comprised in the gene expression vector is a promoter having an activity to induce gene expression in a somatic cell as the subject of the initialization. The somatic cell into which the gene expression vector has been introduced is in principle a mammalian cell, particularly a human-derived cell; and therefore, the promoter may be a promoter capable of expressing a downstream gene in the cell. Examples of the promoter include a CMV promoter (CMV-IE promoter), a SV40 early promoter, a RSV promoter, an HSV-TK promoter, an EF1α promoter, a Ub promoter, a metallothionein promoter, an SRα promoter and a CAG promoter. Other examples thereof include a temperature-controllable heat shock promoter, and inducible promoters such as a tetracycline responsive promoter that can be controlled by the presence/absence of tetracycline.

In the method of the present invention, the gene expression vector may comprise, as an optional component, a regulatory sequence other than the above-mentioned promoter, a selective marker gene, and/or a reporter gene.

In the method of the present invention, regulatory sequences other than promoters that can be comprised in the gene expression vector include an expression regulatory sequence, an intron sequence, a nuclease recognition sequence and a replication origin sequence. Examples of the expression regulatory sequence include expression regulatory sequences such as an enhancer, a ribosome-binding sequence, a terminator and a poly A addition signal. Examples of the nuclease recognition sequence include a restriction enzyme recognition sequence, a IoxP sequence that is recognized by Cre recombinaze, a sequence that is targeted by an artificial nuclease such as ZFN or TALEN, and a sequence that is targeted by a CRISPR/Cas9 system. Examples of the replication origin sequence include an SV40 replication origin sequence.

For example, a nuclease recognition sequence can be introduced to the front or the rear of a coding region of an initialization factor in the gene expression vector in the method of the present invention. In that case, after initialization of a somatic cell is completed, introduction of a nuclease enables removal of a coding region for an initialization factor.

In the method of the present invention, the selective marker gene that can be comprised in the gene expression vector is a selective marker gene that can select a somatic cell having the gene expression vector introduced thereinto. Specific examples of the selective marker gene include drug resistance genes such as an ampicillin resistance gene, a kanamycin resistance gene, a tetracycline resistance gene, a chloramphenicol resistance gene, a neomycin resistance gene, a puromycin resistance gene and a hygromycin resistance gene.

In the method of the present invention, the reporter gene that can be comprised in the gene expression vector is a gene encoding a reporter capable of identifying a somatic cell having a gene expression vector introduced thereinto. Examples of the reporter gene include a gene encoding a fluorescent protein such as GFP or RFP, a luciferase gene.

### (Initialization Gene)

In the method of the present invention, the initialization inducer comprises, as an essential component, one or more (1) initialization genes incorporated into a gene expression vector. In addition, the initialization inducer may optionally include one or more (2) cofactors. Hereinafter, (1) and (2) will be specifically described.

(1) With respect to the initialization gene, an initialization inducer in the method of the present invention comprises one or more initialization genes. An initialization factor encoded by an initialization gene is not limited as long as it is a factor capable of inducing initialization of a somatic cell. Examples thereof include OCT3/4, SOX2, C-MYC and KLF, and associated factors of any of them. Examples of the associated factors include SOX1, SOX3, SOX15, SOX18, a T58A mutant of C-MYC, N-MYC and L-MYC, and KLF1, KLF2, KLF4 and KLF5. These initialization factors may be either of a protein corresponding to the initialization factor and a peptide fragment thereof and, particularly, at least one initialization factor selected from the group consisting of OCT3/4, KLF4 and L-MYC can be suitably used. That is, the initialization inducer preferably comprises at least one gene selected from the group consisting of an OCT3/4 gene, a KLF4 gene and an L-MYC gene.

In the method of the present invention, the number of initialization genes comprised in the initialization inducer is not limited. The initialization inducer may comprise, for example, one, two, three, four, five, six or more initialization genes.

When the initialization inducer comprises two or more initialization genes, the two or more initialization genes may be comprised in the same vector or different vectors.

(2) With respect to the initialization auxiliary factor, “initialization auxiliary factor” in the method of the present invention that can be comprised as an optional component in the initialization inducer refers to a factor that is one other than those listed in the above (1), is not essential for initialization induction of a somatic cell, and can increase the efficiency of initialization induction when it is introduced in a somatic cell. Examples thereof include NANOG, NR5A2, LIN28A, LIN28B, LIN41, GLIS1, TBX3, HMGA2, FOXH1, mir-302, mir-367, mir-106a, mir-363, shRNA or siRNA against TP53, dominant-negative TP53, and shRNA or siRNA against P21.

In the step I of the present invention, a method for introducing into a somatic cell an initialization gene comprised in the initialization inducer is not particularly limited, and a transfection method may be appropriately selected depending on the type of a gene (plasmid vector, viral vector, etc.). An initialization gene can be introduced into a somatic cell by, for example, viral infection, lipofection method, liposome method, electroporation method, calcium phosphate method or DEAE-Dextran method. In addition to these, known gene transfer method (transformation methods) in the art described in Green & Sambrook, 2012, Molecular Cloning: A laboratory Manual Fourth Ed., Cold Spring Harbor Laboratory Press, Cold Spring Harbor, New York and others may be used. For introduction of an initialization gene, a somatic cell into which the initialization gene has been introduced may be present in, for example, a liquid medium, or a solution special for electroporation, but this is not particularly limited.

### 1-3-2. Initialization and Amplification-Culture Step (Step II)

The step II in the method of the present invention is a step of performing initialization and amplification culture of a cell into which an initialization gene has been introduced in the step I. In the present invention, it is preferable to proceed to the step II within 5 days at the longest, particularly within 3 days, and further within 1 day after an initialization gene is introduced in the step I. In the steps II of the present invention, cell culture is performed in a liquid medium under a suspension culture condition. In the step II, the liquid medium to be used comprises at least one of a protein kinase Cβ (PKCβ) inhibitor and a WNT inhibitor.

Hereafter, an animal cell culture method to be used in the present step and the subsequent culture step will be exemplified and explained, but it is not limited thereto.

### (Culture Vessel)

A vessel the inner surface of which is less adhesive to cells is preferred as the culture vessel to be used for culture. Examples of the vessel the inner surface of which is less adhesive to cells include a plate subjected to hydrophilic surface treatment with a biocompatible substance. For example, a Nunclon™ Sphera (Thermo Fisher Scientific K.K.) can be used as the culture vessel.

The shape of the culture vessel is not limited, and examples of the culture vessel include dish-form, flask-form, well-form, bag-form, and spinner-flask-form culture vessels.

The capacity of the culture vessel to be used is not limited, and an appropriate capacity can be selected; however, it is preferable that the lower limit of the area of the bottom of a part to comprise medium in plain view be 0.32 cm² or larger, 0.65 cm² or larger, 1.9 cm² or larger, 3.0 cm² or larger, 3.5 cm² or larger, 9.0 cm² or larger, or 9.6 cm² or larger, and the upper limit thereof be 1000 cm² or smaller, 500 cm² or smaller, 300 cm² or smaller, 150 cm² or smaller, 75 cm² or smaller, 55 cm² or smaller, 25 cm² or smaller, 21 cm² or smaller, or 10 cm² or smaller.

### (PKCβ Inhibitor and WNT Inhibitor)

The step is characterized by culturing in a medium comprising a PKCβ inhibitor and/or a WNT inhibitor defined in the above section “1-2. Definition of terms.” The type of the medium is not limited as long as it is a medium that comprises a PKCβ inhibitor and/or a WNT inhibitor and can grow and/or maintain cells.

A PKCβ inhibitor may be comprised in a medium either alone or in combination of two or more of different ones. A WNT inhibitor may be comprised in a medium either alone or in combination of two or more different ones. Regarding the PKCβ inhibitor and the WNT inhibitor, the PKCβ inhibitor alone may be comprised in the medium, the WNT inhibitor alone may be comprised therein, or both of them may be comprised therein. Preferably, both of the PKCβ inhibitor and the WNT inhibitor are comprised.

The lower limit of the concentration of the PKCβ inhibitor is not particularly limited and can be determined depending on the range that suppresses the adherence of the above initialized cells.

The final concentration of the PKCβ inhibitor in the liquid medium can be, for example, 25 nM or more, 30 nM or more, 50 nM or more, 80 nM or more, 100 nM or more, 150 nM or more, 200 nM or more, 500 nM or more, 700 nM or more, 1 μM or more, 3 μM or more, 5 M or more, and 10 μM or more.

The upper limit of the concentration of the PKCβ inhibitor in the medium is not limited, and can be determined with considering the range that suppresses the adherence of the initialized cells, the solubility of the PKCβ inhibitor, and others.

The final concentration of the PKCβ inhibitor in the liquid medium can be, for example, 15 μM or less, 10 μM or less, 5 μM or less, 3 μM or less, 1 μM or less, 700 nM or less, 500 nM or less, 200 nM or less, 150 nM or less, 100 nM or less, 80 nM or less, 50 nM or less, and 30 nM or less.

Note that when a PKCβ inhibitor is comprised in a composition and is added to a medium, the concentration of the PKCβ inhibitor in the composition can be determined so as to fall within the above ranges when the composition is added to the medium. If the composition is 2-fold diluted for use, for example, the lower limit of the PKCβ inhibitor in the composition can be 50 nM or more, 60 nM or more, 100 nM or more, 160 nM or more, 200 nM or more, 300 nM or more, 400 nM or more, 1 μM or more, 1.4 μM or more, 2 μM or more, 6 μM or more, 10 μM or more, and 20 μM or more.

The upper limit of the PKCβ inhibitor in the composition is not limited, and can be the solubility of the PKCβ inhibitor. Specifically, the upper limit of the PKCβ inhibitor in the composition can be 200 mM.

The lower limit of the concentration of the WNT inhibitor is not limited, and can be determined with considering the range that suppresses the adherence of the initialized cells.

The final concentration of the WNT inhibitor in the liquid medium can be, for example, 90 nM or more, 100 nM or more, 150 nM or more, 200 nM or more, 300 nM or more, 400 nM or more, 500 nM or more, 600 nM or more, 700 nM or more, 800 nM or more, 900 nM or more, 1 μM or more, 1.5 μM or more, 3 μM or more, 5 μM or more, 10 μM or more, 15 μM or more, 30 μM or more, and 35 M or more.

The upper limit of the concentration of the WNT inhibitor is not limited, and can be determined with considering the range that suppresses the adherence of the initialized cells, the solubility of the WNT inhibitor, and others.

The final concentration of the WNT inhibitor in the liquid medium can be, for example, 40 μM or less, 35 M or less, 30 μM or less, 15 μM or less, 10 μM or less, 5 μM or less, 3 μM or less, 1.5 μM or less, 1 μM or less, 900 nM or less, 800 nM or less, 700 nM or less, 600 nM or less, 500 nM or less, 400 nM or less, 300 nM or less, 200 nM or less, 150 nM or less, and 100 nM or less.

In the case that the WNT inhibitor is comprised in a composition and is added to a medium, the concentration of the WNT inhibitor in the composition can be determined so as to fall within the above range when the composition is added to medium. If the composition is 2-fold diluted for use, for example, the lower limit of the TNKS inhibitor can be 180 nM or more, 200 nM or more, 300 nM or more, 400 nM or more, 600 nM or more, 800 nM or more, 1 μM or more, 1.2 μM or more, 1.4 μM or more, 1.6 M or more, 1.8 μM or more, 2 μM or more, 3 M or more, 6 μM or more, 10 μM or more, 20 μM or more, 30 μM or more, 60 μM or more, and 70 μM or more.

The upper limit of the WNT inhibitor in the composition is not limited, and can be the solubility of the WNT inhibitor. Specifically, the upper limit of the WNT inhibitor in the composition can be 113 mM.

When a PKCβ inhibitor and a WNT inhibitor are allowed to coexist in a liquid medium, the lower limit of the ratio of the concentration of the PKCβ inhibitor and the WNT inhibitor in the liquid medium is not particularly limited, and can be, for example, 167:1 or higher, 111:1 or higher, 56:1 or higher, 33:1 or higher, 11:1 or higher, 7.8:1 or higher, 5.6:1 or higher, 2.2:1 or higher, 1.7:1 or higher, or 1.1:1 or higher. The upper limit of the ratio of the concentrations of the PKCβ inhibitor and the WNT inhibitor comprised in the liquid medium is not limited, and can be, for example, 1:1600 or lower, 1:1400 or lower, 1:1200 or lower, 1:600 or lower, 1:400 or lower, 1:200 or lower, 1:120 or lower, 1:60 or lower, 1:40 or lower, 1:36 or lower, 1:32 or lower, 1:28 or lower, 1:24 or lower, 1:20 or lower, 1:16 or lower, 1:12 or lower, 1:8 or lower, 1:6 or lower, or 1:4 or lower. The ratio of the concentrations of the PKCβ inhibitor and the WNT inhibitor comprised in the liquid medium is not limited, and can be, for example, in the range of 167:1 or higher and 1:1600 or lower. The ratio of the concentrations of the PKCβ inhibitor and the WNT inhibitor comprised in the liquid medium can be in the range of 111:1 or higher and 1:1600 or lower, in the range of 56:1 or higher and 1:1600 or lower, in the range of 33:1 or higher and 1:1600 or lower, in the range of 11:1 or higher and 1:1600 or lower, in the range of 7.8:1 or higher and 1:1600 or lower, in the range of 5.6:1 or higher and 1:1600 or lower, in the range of 2.2:1 or higher and 1:1600 or lower, in the range of 1.7:1 or higher and 1:1600 or lower, and in the range of 1.1:1 or higher and 1:1600 or lower. Moreover, the ratio of the concentrations of the PKCβ inhibitor and the WNT inhibitor comprised in the liquid medium can be in the range of 167:1 or higher and 1:1400 or lower, in the range of 167:1 or higher and 1:1200 or lower, in the range of 167:1 or higher and 1:600 or lower, in the range of 167:1 or higher and 1:400 or lower, in the range of 167:1 or higher and 1:200 or lower, in the range of 167:1 or higher and 1:120 or lower, in the range of 167:1 or higher and 1:60 or lower, in the range of 167:1 or higher and 1:40 or lower, in the range of 167:1 or higher and 1:36 or lower, in the range of 167:1 or higher and 1:32 or lower, in the range of 167:1 or higher and 1:28 or lower, in the range of 167:1 or higher and 1:24 or lower, in the range of 167:1 or higher and 1:20 or lower, in the range of 167:1 or higher and 1:16 or lower, in the range of 167:1 or higher and 1:12 or lower, in the range of 167:1 or higher and 1:8 or lower, in the range of 167:1 or higher and 1:6 or lower, and in the range of 167:1 or higher and 1:4 or lower.

### (Medium)

As described in the section “1-2. Definition of terms,” a preferable medium to be used in the present step is a liquid medium comprising at least one selected from the group consisting of L-ascorbic acid, insulin, transferrin, selenium, and sodium hydrogen carbonate. Another preferable medium to be used in the present invention is a liquid medium comprising at least one growth factor (preferably FGF2 and/or TGF-β1). A particularly preferable medium is a DMEM/F12 medium that: comprises L-ascorbic acid, insulin, transferrin, selenium, and sodium hydrogen carbonate, and at least one growth factor (preferably FGF2 and TGF-β1); and is free of serum. As the medium to be used in the present step, preferred is a liquid medium comprising a ROCK inhibitor (preferably Y-27632). Further, the medium to be used in the present step is preferably a non-gelled medium. That is, a solid medium or a semi-solid medium is not used, and a liquid medium having a high fluidity is preferably used. In the present specification, “non-gelled medium” or a liquid medium indicates a medium having a viscosity of 0.90 mPa-S or less, and preferably 0.85 mPa-S or less.

### (Composition)

When a PKCβ inhibitor and/or a WNT inhibitor are added as a composition comprising them to a medium, examples of additional components combined with the PKCβ inhibitor and/or the WNT inhibitor as active ingredients for the composition include a carrier. The carrier comprises a solvent and/or an excipient.

Examples of the solvent include water, buffers (including PBS), physiological saline, and organic solvents (DMSO, DMF, xylene, lower alcohols).

Examples of the excipient include antibiotics, buffering agents, thickeners, coloring agents, stabilizers, surfactants, emulsifying agents, antiseptics, preservatives, and antioxidants. Applicable antibiotics include, but are not limited to, penicillin, streptomycin, and amphotericin B. Examples of buffering agents include phosphate buffer, Tris-hydrochloric acid buffer, and glycine buffer. Examples of thickeners include gelatin and polysaccharides. Examples of coloring agents include phenol red. Examples of stabilizers include albumin, dextran, methylcellulose, and gelatin. Examples of surfactants include cholesterol, alkyl glycoside, alkyl polyglucoside, alkyl monoglyceryl ether, glucoside, maltoside, neopentyl glycols, polyoxyethylene glycols, thioglucoside, thio maltoside, peptide, saponin, phospholipid, fatty acid sorbitan ester, and fatty acid diethanolamide. Examples of emulsifying agents include glycerin fatty acid ester, sorbitan fatty acid ester, propylene glycol fatty acid ester, and sucrose fatty acid ester. Examples of antiseptics include aminoethylsulfonic acid, benzoic acid, sodium benzoate, ethanol, sodium edetate, agar, dl-camphor, citric acid, sodium citrate, salicylic acid, sodium salicylate, phenyl salicylate, dibutylhydroxytoluene, sorbic acid, potassium sorbate, nitrogen, dehydroacetic acid, sodium dehydroacetate, 2-naphthol, white soft sugar, honey, isobutyl para-hydroxybenzoate, isopropyl para-hydroxybenzoate, ethyl para-hydroxybenzoate, butyl para-hydroxybenzoate, propyl para-hydroxybenzoate, methyl para-hydroxybenzoate, I-menthol, and Eucalyptus oil. Examples of preservatives include benzoic acid, sodium benzoate, ethanol, sodium edetate, dried sodium sulfite, citric acid, glycerin, salicylic acid, sodium salicylate, digutylhydroxytoluene, D-sorbitol, sorbic acid, potassium sorbate, sodium dehydroacetate, isobutyl para-hydroxybenzoate, isopropyl para-hydroxybenzoate, ethyl para-hydroxybenzoate, butyl para-hydroxybenzoate, propyl para-hydroxybenzoate, methyl para-hydroxybenzoate, propylene glycol, and phosphoric acid. Examples of antioxidants include citric acid, citric acid derivatives, vitamin C and derivatives thereof, lycopene, vitamin A, carotenoids, vitamin B and derivatives thereof, flavonoids, polyphenols, glutathione, selenium, sodium thiosulfate, vitamin E and derivatives thereof, α-lipoic acid and derivatives thereof, pycnogenol, flavangenol, superoxide dismutase (SOD), glutathione peroxidase, glutathione-S-transferase, glutathione reductase, catalase, ascorbate peroxidase, and mixtures of them.

The composition may comprise one or more growth factors. Examples of the growth factors include FGF2 and TGF-β1.

### (Culture)

As long as concentrations of a PKCβ inhibitor and a WNT inhibitor in the medium at the beginning of the present step fall within the above ranges, a method for adding the PKCβ inhibitor and/or the WNT inhibitor is not particularly limited. For example, the medium may be prepared by directly administering one or more PKCβ inhibitors and/or WNT inhibitors to the medium such that each concentration in total falls within the corresponding concentration range shown above.

In the step II, somatic cells into which an initialization factor has been introduced in the step I are used. When the somatic cells into which an initialization factor has been introduced are in a suspension state, suspension culture can be continued in the step II directly from the step I. When the cells into which an initialization factor has been introduced are adhered to a container or a substrate, they are detached by enzymatic treatment, etc. and then, provided for the step II. In terms of the simplicity of the step, it is preferred to continue suspension culture in the step II directly from the step I wherein somatic cells into which an initialization factor has been introduced are not adhered to a container or a substrate. The volume of a medium or culture solution can be appropriately adjusted for a culture vessel to be used. When a 96-well plate (area of well bottom per well in plain view: 0.35 cm²) is used, for example, the volume per well can be 0.05 mL or more, and 0.3 mL or less, and preferably about 0.1 mL. If a 12-well plate (area of well bottom per well in plan view: 3.5 cm²) is used, for example, the volume per well can be 0.5 mL or more, 1.5 mL or less, and preferably approximately 1.3 mL. If a 6-well plate (area of well bottom per well in plan view: 9.6 cm²) is used, the lower limit of the volume per well can be 1.5 mL or more, 2 mL or more, or 3 mL or more, and the upper limit thereof can be 6.0 mL or less, 5 mL or less, or 4 mL or less. When a 30-mL spinner-flask (a spinner-flask having a capacity of 30 mL) is used, the lower limit of the volume per vessel can be 20 mL or more, 25 mL or more, or 30 mL or more; and the upper limit thereof can be 40 mL or less, 35 mL or less and 30 mL or less. If a 125-mL Erlenmeyer flask (an Erlenmeyer flask having a capacity of 125 mL) is used, the lower limit of the volume per vessel can be 10 mL or more, 15 mL or more, 20 mL or more, 25 mL or more, or 30 mL or more, and the upper limit thereof can be 50 mL or less, 45 mL or less, or 40 mL or less. If an Erlenmeyer flask having a capacity of 500 mL is used, the lower limit of the volume per vessel can be 100 mL or more, 105 mL or more, 110 mL or more, 115 mL or more, or 120 mL or more, and the upper limit thereof can be 150 mL or less, 145 mL or less, 140 mL or less, 135 mL or less, 130 mL or less, or 125 mL or less. If an Erlenmeyer flask having a capacity of 1000 mL is used, the lower limit of the volume per vessel can be 250 mL or more, 260 mL or more, 270 mL or more, 280 mL or more, or 290 mL or more, and the upper limit thereof can be 350 mL or less, 340 mL or less, 330 mL or less, 320 mL or less, or 310 mL or less. When a stirring reactor having a capacity of 1000 mL is used, the lower limit of the volume per vessel can be 300 mL or more, 310 mL or more, or 320 mL or more; and the upper limit thereof can be 1000 mL or less, 900 mL or less, 800 mL or less, 700 mL or less, or 600 mL or less. If a disposable culture bag, for example, having a capacity of 2 L is used, the lower limit of the volume per bag can be 100 mL or more, 200 mL or more, 300 mL or more, 400 mL or more, 500 mL or more, 600 mL or more, 700 mL or more, 800 mL or more, 900 mL or more, or 1000 mL or more, and the upper limit thereof can be 2000 mL or less, 1900 mL or less, 1800 mL or less, 1700 mL or less, 1600 mL or less, 1500 mL or less, 1400 mL or less, 1300 mL or less, 1200 mL or less, or 1100 mL or less. If a disposable culture bag having a capacity of 10 L is used, the lower limit of the volume per bag can be 500 mL or more, I L or more, 2 L or more, 3 L or more, 4 L or more, or 5 L or more, and the upper limit thereof can be 10 L or less, 9 L or less, 8 L or less, 7 L or less, or 6 L or less.

The step II may include a medium exchange procedure and a procedure of seeding in a new medium (subculture). In a subculture, the density of cells to be seeded in a new medium (seeding density) can be appropriately adjusted in view of culture time, the condition of cells after culture, and the number of cells needed after culture. Typically, the seeding density can be, though not limited to, in such a range that the lower limit is 0.001×10³ cells/mL or more, 0.01×10⁵ cells/mL or more, 0.1×10⁵ cells/mL or more, 1×10⁵ cells/mL or more, or 2×10⁵ cells/mL or more; and the upper limit is 20×10³ cells/mL or less, or 10×10⁵ cells/mL or less.

There is no limitation to the culture conditions including culture temperature, time, and CO₂ concentration. Culture can be performed with any of conventional methods in the art. For the culture temperature, for example, the lower limit can be 20° C. or higher or 35° C. or higher, and the upper limit can be 45° C. or lower or 40° C. or lower, though the culture temperature is preferably 37° C. The culture time can be in such a range that the lower limit is 0.5 hours or longer or 6 hours or longer, and the upper limit is 7 days or shorter, 120 hours or shorter, 96 hours or shorter, 72 hours or shorter, or 48 hours or shorter. For the CO₂ concentration in culture, the lower limit can be 0.5% or more, 1% or more, 2% or more, 3% or more, 4% or more or 4.5% or more, and the upper limit can be 10% or less or 5.5% or less, though the CO₂ concentration in culture is preferably 5%. Medium exchange can be performed at an appropriate frequency. The frequency of medium exchange varies among cell types to be cultured, and medium exchange can be performed, for example, one or more times per 5 days, one or more times per 4 days, one or more times per 3 days, one or more times per 2 days, one or more times per day, or two or more times per day, though the frequency is not limited thereto. Medium exchange can be performed in such a manner that cells are collected with the same method as in the collection step, fresh medium is then added, and the cell aggregate is gently dispersed, and then cultured again. Alternatively, a certain amount of medium liquid is always and continuously removed by suction, and a new medium is continuously added. The frequency of and method or the like for medium exchange are not limited to the above frequency and method, and an optimum method can be appropriately employed.

The timing of termination of culture and the timing of medium exchange may be determined, for example, on the basis of lactic acid concentration in medium. Lactic acid is produced by cells during culture, and accumulated in medium. Lactic acid produced by cells or lactic acid originally comprised in medium is known to damage cells, thereby inhibiting the maintenance of the undifferentiation of iPS cells to lead to adverse effects on proliferation such as lowered cell proliferative capacity, in particular, after subculture. The inhibitory effect of lactic acid on maintenance of undifferentiation and the lowering effect of lactic acid on cell proliferative capacity can be avoided by determining the timing of termination of culture and/or the timing of medium exchange on the basis of lactic acid concentration in medium.

In the present step, initialized cells are suspension-cultured in the presence of a PKCβ inhibitor and/or a WNT inhibitor and this can reduce the above effects of lactic acid. In other words, in suspension culture of cells in the presence of a PKCβ inhibitor and/or WNT inhibitor, the undifferentiation can be maintained or the cell proliferative capacity can be maintained even under higher lactic acid concentration than in common cell culture. Specifically, culture can be performed in a favorable manner even when the lactic acid concentration in medium reaches 5 mM or more. Moreover, culture can be performed in a favorable manner even when the lactic acid concentration in medium reaches 7 mM or more, 9 mM or more, 10 mM or more, 11 mM or more, or 12 mM or more.

It is preferable that the lactic acid concentration in medium be up to 15 mM. In particular, it is preferable that the lactic acid concentration in medium be 14 mM or less, 13 mM or less, 12 mM or less, or 10 mM or less. With the lactic acid concentration in medium being within the range, significant lowering of the pH of medium can be prevented, and the above-described effects on cells can be avoided.

As described above, in suspension culture of initialized cells in the presence of a PKCβ inhibitor and/or a WNT inhibitor, the timing of termination of culture and the timing of medium exchange can be set to a timing when the lactic acid concentration in medium reaches, for example, 15 mM, 14 mM or less, 13 mM or less, 12 mM or less, or 10 mM or less. In another embodiment, the timing of termination of culture and the timing of medium exchange may be set to a time at which the lactic acid concentration in medium is lower than the range.

### (Suspension Culture)

The present step employs suspension culture. Either of flow culture and static culture may be employed as long as they enable suspension culture, but flow culture is preferred. “Flow culture” is to culture under a condition where a medium is flown. In the case of flow culture, preferred is a method that promote the aggregation of cells in a single cell state and allows a medium to flow for prevention of excessive aggregation. Examples of such a culture method include a suspension rotational method, a suspension rocking method, a suspension stirring method, or combinations thereof. In particular, a suspension rotational method or a suspension stirring method can be suitably used. In contrast, “static culture” is to culture in a state where a medium is left to stand in a culture vessel and is usually used for adherent culture.

“Suspension rotational method” (including a shaking culture method) refers to a method of culturing under conditions allowing a medium to flow so that cells can gather to one point by stress (centrifugal force, centripetal force) due to a rotational flow. Specifically, rotational culture is performed by rotating a culture vessel comprising medium with cells along a closed trajectory such as a circle, an ellipse, a flattened circle, and a flattened ellipse in a generally horizontal plane.

The rotational speed is not limited, and can be 1 rpm or higher, 10 rpm or higher, 50 rpm or higher, 60 rpm or higher, 70 rpm or higher, 80 rpm or higher, 83 rpm or higher, 85 rpm or higher, or 90 rpm or higher. The rotational speed can be 200 rpm or lower, 150 rpm or lower, 120 rpm or lower, 115 rpm or lower, 110 rpm or lower, 105 rpm or lower, 100 rpm or lower, 95 rpm or lower, or 90 rpm or lower. The amplitude of a shaker to be used for suspension rotational method is not limited, and the lower limit can be, for example, 1 mm or larger, 10 mm or larger, 20 mm or larger, or 25 mm or larger. The upper limit can be, for example, 200 mm or smaller, 100 mm or smaller, 50 mm or smaller, 30 mm or smaller, or 25 mm or smaller. Likewise, the rotational radius in suspension rotational method is not limited, and preferably set in such a manner that the amplitude falls within the above range. The lower limit of the rotational radius is, for example, 5 mm or larger or 10 mm or larger, and the upper limit thereof can be, for example, 100 mm or smaller or 50 mm or smaller. In particular, it is advantageous that adoption of the above ranges for rotational conditions is likely to allow produced cell aggregates to have proper sizes.

“Suspension rocking method” refers to a method of culturing under conditions to provide a medium with a rocking flow through linear reciprocating motion such as rocking stirring. Specifically, suspension rocking method is performed by rocking a culture vessel comprising medium with cells in a plane generally perpendicular to the horizontal plane. The rocking speed is not limited, and rocking can be performed in such a manner that the lower limit as one reciprocating motion is regarded as one cycle is, for example, 2 cycles or more, 4 cycles or more, 6 cycles or more, 8 cycles or more, or 10 cycles or more per minute, and the upper limit is 15 cycles or less, 20 cycles or less, 25 cycles or less, or 50 cycles or less per minute. In rocking, it is preferable to slightly incline the culture vessel to the vertical plane, in other word, provide the culture vessel with a guide angle. The rocking angle is not limited, and the lower limit can be, for example, 0.10 or larger, 2° or larger, 4° or larger, 6° or larger, or 8° or larger, and the upper limit can be 20° or smaller, 180 or smaller, 150 or smaller, 12° or smaller, or 10° or smaller. In particular, it is advantageous that adoption of the above ranges for rocking conditions is likely to allow produced cell aggregates to have proper sizes.

Furthermore, culture can be performed with stirring by motion as a combination of the rotation and rocking.

“Suspension stirring method” refers to a method of culturing under conditions involving stirring a medium in a culture vessel by using stirring means such as a stirrer bar and a stirring blade while the culture vessel is left to stand. For example, suspension stirring method can be achieved by using a spinner-flask-like culture vessel equipped with a stirring blade. Such culture vessels are commercially available, and they may be used. In the case of a commercially available spinner-flask-like culture vessel, a cell culture composition in an amount recommended by the manufacturer can be used in a favorable manner.

In the “suspension stirring method”, it is preferable to control the shear stress applied to cells during culture. Generally, most animal cells including iPS cells are more susceptible to physical stress than other cells. Accordingly, if the shear stress applied to cells in stirring culture is excessively high, the cells may be physically damaged to result in lowered proliferative capacity, or, for iPS cells, failure in maintaining the undifferentiation. The number of rotations of the stirring blade is not particularly limited, but the lower limit thereof can be 1 rpm or more, 10 rpm or more, 30 rpm or more, 50 rpm or more, or 70 rpm or more, 90 rpm or more, 110 rpm or more, or 130 rpm or more. The upper limit can be 200 rpm or lower or 150 rpm or lower.

The shear stress applied to cells in suspension stirring method is not limited, but depends, for example, on the blade tip speed. The blade tip speed is the circumferential speed of the tip of the stirring blade, and determined by blade diameter [m]×pi×rotational frequency [rps]=blade tip speed [m/s]. If the tip shape of the stirring blade gives a plurality of blade diameters, the largest length can be employed.

In particular, the effects of applied shear stress can be reduced in suspension culture of cells in the presence of a PKCβ inhibitor and/or a WNT inhibitor. In other words, in suspension culture of iPS cells in the presence of a PKCβ inhibitor and/or a WNT inhibitor, the undifferentiation can be maintained or the cell proliferative capacity can be maintained even under conditions involving application of higher shear stress than in common cell culture. Specifically, stirring culture can be performed with the undifferentiation of iPS cells maintained even when the blade tip speed is a very high value of 0.23 m/s or higher.

The blade tip speed is preferably 0.05 m/s or higher, preferably 0.08 m/s or higher, preferably 0.10 m/s or higher, preferably 0.13 m/s or higher, preferably 0.17 m/s or higher, preferably 0.20 m/s or higher, preferably 0.23 m/s or higher, preferably 0.25 m/s or higher, and preferably 0.30 m/s or higher. With the blade tip speed being within the range, excessive cell-to-cell aggregation can be prevented with the undifferentiation of iPS cells maintained.

The blade tip speed is preferably 1.37 m/s or lower, preferably 1.00 m/s or lower, preferably 0.84 m/s or lower, preferably 0.50 m/s or lower, preferably 0.42 m/s or lower, preferably 0.34 m/s or lower, and preferably 0.30 m/s or lower. Adoption of the above ranges for blade tip speed can stabilize the flowing state of a medium in a culture system while maintaining the undifferentiation of iPS cells.

As described above, the step II may include a procedure of subculture, and the subculture can be performed with a modification of culture scale, preferably an increase of culture scale (scale-up). For the subculture with a modification of culture scale, the numbers of stirring blade rotations of stirring means before and after the modification can be determined such that powers required for stirring or blade tip speeds per volume are the same, preferably different within less than ±10%, and more preferably less than ±5% in line with the culture scale. At the time of initializing or establishing iPS cells, a smaller number of cells are cultured to gradually proliferate and a large amount of cells are obtained; thus, an increase of culture scale along with progression of culture is needed. At that time, if powers required for stirring or blade tip speeds per volume of a culture solution are always determined to be the same, cells can be cultured in the same culture environment regardless of the size of scale. For example, when the culture scale is increased as described above, for analogous modification of the culture scale, the number of stirring blade rotations can be determined using the Pv constant formula. Pv represents a power required for stirring per unit volume; and when Pv is kept the same, stirring culture with the same condition can be performed between different scales. Pv constant formula can be represented by: number of rotations per unit time [rpm or rps]×(blade diameter [m])^{2/3}=constant.

To what degree the number of cells is increased and to what state cells are adjusted in the present step can be appropriately determined according to the type of cells to be cultured, the type of medium, and the culture conditions. The present step can provide initialized cells, that is a cell population comprising iPS cells.

### (Collection)

The step II preferably includes a procedure of separating cells from a culture solution with a conventional method and collecting a cell population after culture under the above conditions. At this time, the cell population to be collected usually forms a cell aggregate. In the method of the present invention, the step II preferably includes a procedure of collecting formed cell aggregates.

The cells after culture of the step II are usually present in a suspended state in a culture solution in the form of a cell aggregate. Thus, cells can be collected by removing a liquid component of supernatant by leaving to stand or through centrifugation. Alternatively, cells may be collected with a filtration filter, a hollow fiber separation membrane or the like. When the liquid component is removed by leaving to stand, a vessel comprising the culture solution is left to stand for about 5 minutes and the supernatant may be removed while precipitated cells or cell aggregates are left unremoved. Centrifugation can be performed with such a centrifugal acceleration and treatment time that cells are not damaged by the centrifugal force. For example, the lower limit of the centrifugal acceleration is not limited as long as cells can be precipitated, and can be, for example, 100×g or higher, 300×g or higher, 800×g or higher, or 1000×g or higher. The upper limit can be such a speed that cells are nod damaged or hardly damaged by the centrifugal force, and can be, for example, 1400×g or lower, 1500×g or lower, or 1600×g or lower. The lower limit of the treatment time may be any time that allows cells to be precipitated by the centrifugal acceleration without limitation, and can be, for example, 30 seconds or longer, 1 minute or longer, 3 minutes or longer, or 5 minutes or longer. The upper limit can be such a time that cells are not damaged or hardly damaged by the centrifugal acceleration, and can be, for example, 10 minutes or shorter, 8 minutes or shorter, 6 minutes or shorter, or 30 seconds or shorter. If liquid components are removed through filtration, for example, the culture solution is passed through a nonwoven fabric or a mesh filter to remove the filtrate, and the residual cell aggregates can be collected. If liquid components are removed with a hollow fiber separation membrane, for example, cells can be collected by separating the culture solution and the cells with use of an apparatus including a hollow fiber separation membrane such as a Cell Washing Concentration System (KANEKA CORPORATION).

The cells collected can be washed if necessary. A washing method is not limited. As a washing solution, buffer (including PBS buffer), physiological saline or a medium (preferably minimal essential medium) can be used.

### Effect of Step II

The somatic cells to which an initialization gene has been introduced in the step I, are cultured in a medium comprising a PKCβ inhibitor and/or a WNT inhibitor in the step II, and thereby, they can be initialized, that is changed to iPS cells under a suspension culture condition without adherent culture. iPS cells can be produced by the steps I and II; after the step II, the purity of iPS cells in cell aggregates comprising iPS cells obtained after the step II can be increased by any of known methods. The present invention further provides step III and step IV that enables a cell population comprising iPS cell with a high purity to be obtained by a simple method.

### 1-3-3. Sorting Step (Step III)

The step III in the method of the present invention is a step of sorting undifferentiation marker-positive cells from the cells obtained in the step II. In the present specification, “sorting (of cells)” refers to a procedure of separating cells having a certain characteristic from other cells. In the step Ill, it specifically refers to a procedure of separating cells positive for undifferentiation markers from a group of cells negative for the undifferentiation markers. A cell sorting method may conform to a conventional method and is not particularly limited.

### (Single Cell Formation)

The step III is preferably a step of fractionating cells. For that purpose, the step III preferably has a procedure for single-cell formation of collected cells. “Single-cell formation” refers to forming single, free cells by dispersing a cell assembly in which a plurality of cells is adhering to each other or aggregating, such as a monolayer piece of cells and a cell aggregate. In the present step, the number of fractionated cells is not particularly limited, but it is usually 10 or less, preferably 5 or less, 4 or less, 3 or less, or 2 or less; and further, the cells are preferably fractionated single and independent cells.

For single-cell formation, a detaching agent and/or a chelating agent is used. The detaching agent is not limited, and, for example, trypsin, collagenase, Pronase, hyaluronidase, and elastase are applicable, and commercially available Accutase®, Accumax®, TrypLE™ Express Enzyme (Life Technologies Japan Ltd.), TrypLE™ Select Enzyme (Life Technologies Japan Ltd.), Dispase®, and the like are also applicable. If trypsin is used for single-cell formation, for example, the lower limit of the concentration in the solution may be any concentration that allows a cell assembly to be dispersed without limitation, and can be, for example, 0.15 vol % or more, 0.18 vol % or more, 0.20 vol % or more, or 0.24 vol % or more. The upper limit of the concentration in the solution may be any concentration such that cells themselves are not affected, for example, not dissolved, without limitation, and can be 0.30 vol % or less, 0.28 vol % or less, or 0.25 vol % or less. Although the treatment time depends on the concentration of trypsin, the lower limit may be any time that allows a cell assembly to be sufficiently dispersed by the action of trypsin without limitation, and can be, for example, 5 minutes or longer, 8 minutes or longer, 10 minutes or longer, 12 minutes or longer, or 15 minutes or longer. The upper limit of the treatment time may be any time such that cells themselves are not affected, for example, not dissolved by the action of trypsin without limitation, and can be, for example, 30 minutes or shorter, 28 minutes or shorter, 25 minutes or shorter, 22 minutes or shorter, 20 minutes or shorter, or 18 minutes or shorter. When a commercially available detaching agent is used, it may be used at a concentration that allows cells to be dispersed into single forms as shown in the attached protocol. Single-cell formation can be promoted by mild physical treatment after the treatment with a detaching agent and/or a chelating agent. This physical treatment is not limited, and examples thereof include multiple times of pipetting for cells together with solution. In addition, cells may be passed through a strainer or a mesh, as necessary.

The cells subjected to single-cell formation can be collected by removing a supernatant comprising the detaching agent by leaving to stand or through centrifugation. The collected cells may be washed, as necessary. The same centrifugation conditions and washing method as above may be adopted.

### (Single Cell Sorting)

The step III has a procedure of detecting whether the group of collected cells, preferably the group of cells subjected to single-cell formation expresses an undifferentiation marker on the cell surface. Means for detecting an undifferentiation marker on the cell surface is not limited; however, examples thereof include flow cytometry. In the case that a fluorescence-labeled antibody is used as a detection reagent in flow cytometry, if a cell emitting more intense fluorescence than a negative control (isotype control) is detected, the cell is determined as “positive” for the marker. The proportion of cells positive for a fluorescence-labeled antibody as analyzed by flow cytometry is occasionally referred to as the “positive rate”. Any antibody known in the art can be used for such fluorescence-labeled antibodies, and examples thereof include, but are not limited to, antibodies labeled with fluorescein isothiocyanate (FITC), phycoerythrin (PE), or allophycocyanin (APC).

Examples of the undifferentiation markers on the cell surface as the subject of detection include, as described in the section “1-2. Definition of terms,” Alkaline Phosphatase, NANOG, OCT4, SOX2, TRA-1-60, TRA-1-81, TRA-2-49, c-Myc, KLF4, LIN28, SSEA-4 and SSEA-1. In particular, at least one undifferentiation marker selected from the group consisting of SSEA-4, TRA-1-60, TRA-1-81 and TRA-2-49 is preferred. Specifically, a fluorescent labeled antibody that specifically binds to at least one of these undifferentiation markers is used to fluorescently stain cells, and for example, each single cell can be detected and sorted using flow cytometry (single cell sorting).

### Effect of Step III

As described above, the step II and the subsequent step III can implement production and sorting of iPS cells by initializing somatic cells without conducting adherent culture and colony selection by using a feeder cell or a substrate. Further, iPS cells are slightly different from one another in terms of the quality of individual initialized cells due to the difference in the initialization efficiency for each cell; and therefore, performing single cell sorting enables selective culture and amplification of iPS cells with a more stably quality, iPS cells with a high differentiation capacity into a specific cell species or the like.

### 1-3-4. Suspension Culture Step (Step IV)

The step IV in the method of the present invention is a step of performing suspension culture and proliferation of undifferentiation marker-positive cells sorted in the step III to obtain a cell population comprising iPS cells. The cell culture method in the present step basically conforms to the culture method described in the section “1-3-2. Initialization and amplification-culture step (Step II).” Thus, the same explanation of the step IV as that of the step II is omitted herein, and only characteristic points to the present step alone will be described in detail.

### (Cells)

The cells cultured in the present step are single cells positive for undifferentiation markers sorted in the step III. That is, they are cells, on which the expression of known undifferentiation markers such as Alkaline Phosphatase, NANOG, OCT4, SOX2, TRA-1-60, TRA-1-81, TRA-2-49, c-Myc, KLF4, LIN28, SSEA-4 and SSEA-1, and preferably, at least one undifferentiation marker selected from the group consisting of SSES-4, TRA-1-60, TRA-1-81 and TRA-2-49.

### (Medium)

The medium used for the present step may have the same composition as or a different composition from the medium used in the step II, but preferred is a medium comprising at least one selected from the group consisting of a PKCβ inhibitor and a WNT inhibitor. For other optional elements, the same configuration as described in the section “1-3-2. Initialization and amplification-culture step (Step II)” is adopted.

### (Suspension Culture)

In the present step, suspension culture is preferably performed. Thus, a culture method is preferably flow culture that flows a medium. Suspension culture of iPS cells in a medium comprising a PKCβ inhibitor and/or a WNT inhibitor can maintain an undifferentiation state of iPS cells.

### (Detection of Undifferentiation Marker)

In the present step, some of cells in the course of culture can be taken out to check whether the undifferentiated sate is maintained. Whether the undifferentiated state is maintained can be checked by, for example, measuring the expression of an undifferentiation marker expressed on iPS cells taken out during culture. As described above, examples of the undifferentiation marker include Alkaline Phosphatase, NANOG, OCT4, SOX2, TRA-1-60, c-Myc, KLF4, LIN28, SSEA-4 and SSEA-1. Examples of the detection method of these undifferentiation markers include flow cytometry.

When the positive rate of cells taken out during culture for an undifferentiation marker is preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, or more preferably over 90% and 100% or lower, it can be determined that the undifferentiation is maintained.

In the cell population comprising iPS cells in the present step, it is preferable that the proportion of cells positive for particularly OCT4 among undifferentiation markers, be 90% or higher and the proportion of cells positive for NANOG is 90% or higher.

### (Detection of Three Germ Layers)

In the present step, whether the undifferentiated state is maintained can be checked by measuring expression of three germ layer markers (endodermal cell marker, mesodermal cell marker, and ectodermal cell marker) in some cells taken out in the course of culture. Specifically, if all of the positive rates for the endodermal cell marker, mesodermal cell marker, and ectodermal cell marker are preferably 20% or lower, more preferably 10% or lower, more preferably 9%/o or lower, more preferably 8% or lower, more preferably 7% or lower, more preferably 6% or lower, more preferably 5% or lower, more preferably 4% or lower, more preferably 3% or lower, more preferably 2% or lower, more preferably 1% or lower, or more preferably below the detection limit, it can be determined that the undifferentiation is maintained.

The endodermal cell marker is a gene specific to endodermal cells, and examples thereof include SOX17, FOXA2, CXCR4, AFP, GATA4, and EOMES. Endodermal cells differentiate to form tissue of an organ such as the gastrointestinal tract, the lung, the thyroid, the pancreas, and the liver; cells of secretory glands opening in the gastrointestinal tract; the peritoneum, the pleura, the larynx, eustachian tubes, the trachea, the bronchi, the urinary tract (the bladder, most part of the urethra, part of the ureter), and others.

The mesodermal cell marker is a gene specific to mesodermal cells, and examples thereof include T (BRACHYURY), SMA, MESP1, MESP2, FOXF1, HAND1, EVX1, IRX3, CDX2, TBX6, MDXL1, ISL1, SNAI2, FOXCi, and PDGFRa. Mesodermal cells differentiate to form the celom and the mesothelium lining it, muscles, the skeleton, the dermis, connective tissues, the heart, blood vessels (including vascular endothelia), blood (including blood cells), lymphatic vessels, the spleen, the kidney, the ureter, the gonad (testis, uterus, gonad epithelium), and others.

The ectodermal cell marker is a gene specific to ectodermal cells, and examples thereof include FGF5, NESTIN, SOX1, PAX6, and TUJ1. Ectodermal cells differentiate to form the epidermis, the epithelium of the end of the male urethra, hair, nails, skin grands (including mammary glands and sweat glands), sense organs (including the oral cavity, the pharynx, the nose, and the epithelium of the end of the rectum, and salivary glands) the lenses, the peripheral nervous system, and others. A part of the ectoderm invaginates like a groove to form a neural tube in the developmental process, which in term serves as the origin of neurons in the central nervous system including the brain and the spinal cord, and melanocytes.

Expression of those three germ layer markers (endodermal cell marker, mesodermal cell marker, and ectodermal cell marker) can be measured with any detection method in the art. Examples of methods for measuring expression of the three germ layer markers (endodermal cell marker, mesodermal cell marker, and ectodermal cell marker) include, but are not limited to, quantitative real-time PCR analysis, an RNA-Seq method, Northern hybridization, and a hybridization method using a DNA array. In quantitative real-time PCR analysis, the expression level of a marker gene as a target of measurement is converted into a relative expression level to the expression level of an internal standard gene, and the expression level of the marker gene can be evaluated on the basis of the relative expression level. Examples of the internal standard gene can include a glyceraldehyde-3-phosphate dehydrogenase (GAPDH) gene and a 0-actin (ACTB) gene.

(Evaluation of Differentiation Capacity into Three Germ Layers)

Alternatively, the differentiation capacity of some cells taken out during culture into three germ layers may be checked in the present step. A method for evaluating the differentiation capacity into three germ layers is not particularly limited, but the following method may be employed as a specific example. Cells taken out are cultured in an undifferentiation maintenance medium for several days to allow them to form a germ layer, and then, they are cultured under an adherent culture condition using an ordinary medium for animal cell culture such as serum-comprising DMEM medium, and a feeder cell or a substrate. At that time, a PKCβ inhibitor and a WNT inhibitor are not added to the medium. The culture is performed as static culture under ordinary culture conditions, for example, using a 37° C., 5% CO₂ incubator; medium exchange or subculture is performed every 2 or 3 days; and the culture is performed for 16 to 21 days. A cell population obtained by the culture is measured in terms of the expressions of three germ layer markers and thereby, whether a cell maintains a differentiation capacity into three germ layers can be checked. That is, when it is confirmed that cells positive for an endodermal cell marker, cells positive for a mesodermal cell marker and cells positive for an ectodermal cell marker are expressed in the cell population after the culture without a significant imbalance, it is determined that the cells in the cell population maintain the differentiation capacity into three germ layers.

### (Collection)

The step IV preferably has a procedure of separating cells from a culture solution by a conventional method and collecting a cell population. At this time, the cell population to be collected preferably forms cell aggregates. The method of the present invention preferably includes a procedure of collecting formed cell aggregates. Specific means and conditions of the collection procedure of a cell population are equivalent to those described in the section “1-3-2. Initialization and amplification-culture step (Step II).”

The cells collected may be washed if necessary. Details for washing conditions are equivalent to those described in the section “1-3-2. Initialization and amplification-culture step (Step II).”

### 1-3-5. Other Steps

After the above step IV, the method of the present invention may include further optional steps, such as a step of storing obtained iPS cells or a cell population comprising iPS cells, a step of inducing differentiation into specific cells, and a step of re-culturing by thawing a frozen vial.

### (Storage Step)

Regarding the storage step of iPS cells or a cell population comprising iPS cells, as long as iPS cells are stored in an undifferentiated state, specific conditions therefor are not particularly limited, and any known iPS cell storage method can be used. As an iPS cell storage method, usable is a method for suspending and cryopreserving in an existing commercially available cryopreservation solution such as BAMBANKER®hRM (GC LYMPHOTEC Inc.), CP-5E, CP-1 (Kyokuto Pharmaceutical Industrial Co., Ltd.), StemCell Keep (Bio Verde), DAP213 (RIPROCELL Inc.) and STEM-CELLBANKER® (ZENOGEN PHARMA CO., LTD.).

### 2. Cell Population Including iPS Cells

The cell population of the present invention is characterized by comprising iPS cells produced using the method for producing iPS cells (the method of the present invention) described in the section “1. Method for producing induced pluripotent cells (iPS cells).” More specifically, the cell population of the present invention is a cell population obtained by suspension culture of iPS cells obtained by use of the method of the present invention.

In the cell population of the present invention, the rate of cells positive for undifferentiated markers is preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably over 99% and 100%4/or lower. Examples of the undifferentiated markers mentioned herein include Alkaline Phosphatase, NANOG, OCT4, SOX2, TRA-1-60, c-Myc, KLF4, LIN28, SSEA-4 and SSEA-1. In particular, the proportion of cells positive for OCT4 is 90% or higher; and the proportion of cells positive for NANOG is preferably 90% or higher, in particular, 91% or higher, 92% or higher, 93% or higher, 94% or higher, 95% or higher, 96% or higher, 97% or higher, 98% or higher, or 99% or higher.

In the cell population of the present invention, it is preferred that the proportion of cells positive for, especially, OCT4 among undifferentiated markers be 90% or higher, and the proportion of cells positive for NANOG be 90% or higher.

In the cell population of the present invention, positive rates for endodermal, mesodermal and ectodermal cell markers are all preferably 20% or lower, particularly 10% or lower, 9% or lower, 8% or lower, 7% or lower, 6% or lower, 5% or lower, 4% or lower, 3% or lower, 2% or lower, 1% or lower, or a detection limit or lower.

The cell population of the present invention preferably maintains the differentiation capacity into three germ layers. Whether or not the differentiation capacity into three germ layer is confirmed by, for example, a method described in the section “1-3-4. Suspension Culture Step (Step IV).”

The cell population of the present invention can be usually in the form of a cell aggregate. The size of a cell aggregate is not limited, and the lower limit of the size in maximum width in an image observed through a microscope can be 30 μm or larger, 40 μm or larger, 50 μm or larger, 60 μm or larger, 70 μm or larger, 80 μm or larger, 90 μm or larger, or 100 μm or larger. The upper limit can be 1000 μm or smaller, 900 μm or smaller, 800 μm or smaller, 700 μm or smaller, 600 μm or smaller, 500 μm or smaller, 400 μm or smaller, 300 μm or smaller, or 200 μm or smaller. Incidentally, one human iPS cell has a size of about 10 to 30 μm in the maximum width in an observed image. Cell aggregates sized within the range allow even inner cells therein to be well supplied with oxygen and nutrient components, being preferable as an environment for proliferation of cells.

Cell aggregates having the above size ranges are preferably present at a volume-based ratio of 10% or higher, 20% or higher, 30% or higher, 40% or higher, 50% or higher, 60% or higher, 70% or higher, 75% or higher, 80% or higher, 85% or higher, 90% or higher, 95% or higher, 98% or higher, or 100% in the population of the cell aggregates. Populations of cell aggregates including 20% or more of cell aggregates having a size within the above range allow even inner cells in individual cell aggregates to be well supplied with oxygen and nutrient components, being preferable as an environment for proliferation of cells.

It is preferable that the percentage of viable cells (survival rate) in cells constituting the population of cell aggregates be, for example, 50% or higher, 600% or higher, 70% or higher, 80% or higher, or 90% or higher. Cell aggregates having a survival rate within the above ranges can readily maintain the aggregated state, and they are in a state preferable for proliferation of cells.

### 3. Medium for Initialization and Amplification Culture of Somatic Cells

The medium of the present invention for initialization and amplification culture of somatic cells (hereinafter, also referred to as “medium of the present invention”) is characterized in that it is a liquid medium comprising at least one selected from the group consisting of a PKCβ inhibitor and a WNT inhibitor. The medium of the present invention is used in the step II described in the section “1. Method for producing induced pluripotent stem cells (iPS cells).” Further, it is also preferably used in the step IV.

The medium of the present invention is preferably a liquid medium comprising at least one selected from the group consisting of L-ascorbic acid, insulin, transferrin, selenium and sodium hydrogen carbonate. The medium of the present invention is also preferably a liquid medium comprising at least one growth factor (preferably FGF2 and/or TGF-β1). A particularly preferable medium is a DMEM/F12 medium that: comprises L-ascorbic acid, insulin, transferrin, selenium, and sodium hydrogen carbonate, and at least one growth factor (preferably FGF2 and TGF-β31); and is free of serum. Further, the medium of the present invention is preferably a liquid medium comprising a ROCK inhibitor (preferably Y-27632).

The concentration of each component comprised in the medium of the present invention, the preparation condition and other components are equivalent to those described in the sections “1-2. Definition of terms” and “1-3-2. Initialization and amplification-culture step (Step II).”

### 4. Composition for Initialization and Amplification Culture of Somatic Cells

The composition of the present invention for initialization and amplification culture of somatic cells (hereinafter, also referred to as “composition of the present invention”) is characterized in that it comprises at least one selected from the group consisting of a PKCβ inhibitor and a WNT inhibitor to be added to the medium for initialization and amplification culture of somatic cells. The composition of the present invention is a composition prepared with the design for addition to and use in a medium. Addition of the composition of the present invention to an existing medium enables the medium to be a medium suitable for initialization and amplification culture of somatic cells.

The composition of the present invention preferably comprises at least one selected from the group consisting of L-ascorbic acid, insulin, transferrin, selenium and sodium hydrogen carbonate. The composition of the present invention also preferably comprises at least one growth factor (preferably FGF2 and/or TGF-β1). Further, the composition of the present invention is preferably a liquid medium comprising a ROCK inhibitor (preferably Y-27632). Concentrations of the above components in the composition of the present invention can be adjusted such that they will become optimal when the composition is added to a medium.

The concentration of each component comprised in the composition of the present invention, the preparation condition, the form and other components are equivalent to those described in the section “1-3-2. Initialization and amplification-culture step (Step II).” Examples

Hereinafter, a method for producing iPC cells according to the present invention will be described further in detail with reference to Examples; however, the technical scope of the present invention is not limited to Examples described below.

### Example 1: Establishment of iPS Cells Using Sendai Virus

### (1-1) Culture of Blood Cell

A frozen vial of peripheral blood mononuclear cells (AccuCell® Human PBMC purified, 1×10⁷ cells/vial) was taken out from a liquid nitrogen tank, and a lid of the frozen vial was opened and thawed in a safety cabinet. 9 mL of mononuclear culture medium (StemSpan ACF (STEMCELL Technologies, #ST-09805) comprising 100 ng/mL IL-6, 10 ng/mL IL-3, 300 ng/mL SCF, 300 ng/mL TPO, and 300 ng/ml Flt3 ligand) was charged into a 15-mL tube, and the thawed cell suspension was transferred and suspended into the tube and then, was centrifuged at 200 g for 10 minutes. After supernatant was removed, cells were suspended in 4 mL of mononuclear culture medium and seeded into a 6-well plate for suspension culture (Sumitomo Bakelite Co., Ltd., #MS-8006R), and then, rotational culture (90 rpm) was performed for 24 hours at 37° C. and 5% CO₂.

Rotationally cultured mononuclear cells were transferred to a 15-mL tube by a 5-mL pipette and centrifuged (200 g, for 1 minute). Precipitate was washed with 5 mL of PBS and centrifuged again (200 g, for 1 minute). 1 mL of Accutase (NACALAI TESQUE, INC.) was added to the precipitate and the resultant was incubated at 37° C. for 5 minutes. Cells were detached by several times of pipetting using a 1000-μL pipette tip and then, 4 mL of mononuclear culture medium was added. At that time, the number of cells was counted.

### (1-2) Introduction of an Initialization Factor by Sendai Virus and Start of Initialization

A CytoTune™ EX-iPS kit (ID Pharma Co., Ltd.) was used to introduce an inducing factor for iPS into cells. According to the protocol of the kit, hKOS·hKLF4·hL-MYC was diluted with a mononuclear culture medium such that MOI=5 relative to 1×10⁶ cells, a virus solution was prepared. A cell suspension of mononuclear cells with 1×10⁶ cells cultured in (1) was dispensed into two 15-mL tubes, and centrifuged at 200 g for 10 minutes.

Supernatant was removed and cells were suspended in a 2-mL mononuclear culture medium. Into a 15-mL tube having 2 mL of this cell suspension, 2 mL of virus solution and 4 mL of mononuclear culture medium were charged (8 mL in total); and the resultant was gently mixed by a 5-mL plastic pipette. The cell-virus mixture was promptly seeded at 4 mL/well (5×10⁵ cells/well, two wells in a 6-well dish for suspension culture), and rotational culture was started under conditions: 90 rpm, 37° C. and 5% CO₂. On day 3, 5 and 7 of culture, 2.0 mL of StemFit AK02N (Ajinomoto Co., Inc.) (in advance, mixed with A to C solutions and 1% penicillin/streptomycin; hereinafter, expressed as “StemFit medium”) was further added to medium of each well and culture was continued. On day 9, 11, 13 and 15, the medium was half-exchanged (4 mL) with StemFit medium.

### (1-3) Subculture (Initialization Culture)

On day 16 of culture, a suspension culture sample was transferred to a 15-mL tube and centrifuged (200 g, for 1 minute). Precipitate was washed with 5 mL of PBS and centrifuged (200 g, for 1 minute). Precipitate was suspended with Accutase and a sample of the suspension culture was transferred to a 15-mL tube at 37° C. and centrifuged (200 g, for 1 minute). Precipitate was washed with 5 mL of PBS and centrifuged (200 g, for 1 minute); precipitate was suspended again with Accutase and incubated at 37° C. for 5 to 10 minutes. After several times of pipetting with a pipette tip, an equivalent amount of StemFit medium was added and the mixture was centrifuged at 200 g for 2 minutes. Precipitate was suspended with 1 mL of StemFit medium (comprising 10 μM of ROCK inhibitor (Y-27632, Wako Pure Chemical Corporation). All cells were suspended with StemFit medium (+10 M of Y-27632) alone, or StemFit medium+compound (1 μM of Ly333531, 10 μM of IWR1-endo and 10 M of Y-27632) such that the total amount became 4 mL, re-seeding was performed on one well of the 6-well plate. Rotational culture was started (90 rpm, 37° C. and 5% CO₂), and thereafter, half exchange of the medium was performed every 2 days.

On day 23 of culture, halves of cells cultured under each culture condition described above were subcultured to 2 wells in a 6-well plate. At this time, one half was suspended with StemFit medium (+10 μM of Y-27632) alone and the other half was suspended with StemFit medium+compound (1 μM of Ly333531, 10 M of IWR1-endo, and 10 μM of Y-27632) such that the total amount becomes 4 mL; and culture was continued under the same conditions as described previously. Thereafter, medium exchange was performed every 2 days. On day 28, 33, 38 and 43 of culture the subculture was performed in the same manner. For StemFit medium+compound-added cells, proliferation of about 1×10⁶ cells/well was confirmed on day 48 of culture; and seeding therefrom was performed to satisfy 4×10⁵ cells/well. On day 52 of culture, proliferation of about 1.6×10⁶ cells/well was confirmed; and seeding was performed to satisfy 4×10⁵ cells/well. On day 57 of culture, proliferation of about 2.5×10⁶ cells/well confirmed, and seeding was performed to satisfy 4×10⁵ cells/well.

### (1-4) Analysis of Cell Characteristics

### (1-4-1) Evaluation of Expression of Undifferentiation Marker of Cell by Flow Cytometry Analysis

On day 62 of culture, cells were collected and the expression of undifferentiation marker of cell was evaluated by flow cytometry analysis according to the following procedure.

Cell clusters in suspension culture were transferred to a 15-mL tube and centrifuged (200 g, for 1 minute). Supernatant was removed by suction, and precipitated cell clusters were washed with 5 mL of PBS and was further centrifuged (200 g, for 1 minute). Supernatant was removed by suction, 1 mL of Accutase was added to precipitated cell clusters, and incubation was performed at 37° C. for 5 to 10 minutes. Several times of pipetting were performed by a pipette tip for detachment of cells, and an equivalent amount of StemFit medium was added and the mixture was centrifuged at 200 g for 2 minutes. Supernatant was removed by suction, precipitate was suspended by addition of 1 mL of StemFit medium (comprising 10 M of Y-27632), and the number of cells was counted.

After the number of cells was counted, 2.5×10⁵ cells were dispensed to each of three 15-mL tubes for checking (for negative control, etc.), and centrifuged at 200 g for 3 minutes. After centrifugation, supernatant was discarded and 1 to 2 mL of PBS was added. After centrifugation, supernatant was discarded, 250 L of flow cytometry buffer (PBS comprising 0.1% BSA and 0.5 mM EDTA) was added, and pipetting was performed by a pipette tip, so that the cells were suspended.

The following primary antibodies were added to each sample tube, and the mixtures were incubated for about 1 hour on ice with shading.
No antibody
TRA-1-60 (Alexa Fluoro 488 anti-Human TRA-1-60 antibody: Biolegend Inc., #330614) (2.5 μL)

After incubation, 750 μL of flow cytometry buffer was added and the mixture was centrifuged at 200 g for 3 minutes. After centrifugation, supernatant was removed, 500 μL of flow cytometry buffer was added, and pipetting was performed for suspension. A cell suspension was passed through a cell strainer. Thereafter, the samples were maintained on ice with shading.

Measurement on cells in the cell suspension was performed by a flow cytometry apparatus, Cell Sorter (Sony Corporation, SH800). Cells were collected from positive fractions for SSEA-4 and TRA-1-60; and about 1×10⁵ cells were directly collected to a 15-mL tube comprising 4 mL of StemFit medium (comprising 10 μM of Y-27632, 10 μM of IWR1-endo and 1 μM of LY-333531) and were transferred to a 6-well plate for suspension culture; and then, rotational culture was started (90 rpm, 37° C., 5% CO₂). Thereafter, while the state of cells was being observed, subculture is continued every 5 to 6 days, and sequentially, frozen stocks (5×10⁵ to 1×10⁶ cells/vial were suspended in STEM-CELLBANKER) were prepared.

The TRA-1-60 positive rate of cells on day 62 of culture was measured by flow cytometry; and as a result, the TRA-1-60 positive rate was 98.27%. This indicates that iPS cells can be produced under a suspension culture condition.

### (1-4-2) Detection of Undifferentiation Markers by Immunostaining Method

Cell on day 57 of culture were collected and the expression of undifferentiation markers, OCT4, NANOG and SSEA-4 proteins was identified by an immunostaining method. The immunostaining method was carried out according to the following procedure. Suspension culture cell clusters were transferred to a 15 mL tube and centrifuged (200 g, for 1 minute). Supernatant was removed by suction, and precipitated cell clusters were washed with 5 mL of PBS and further centrifuged (200 g, for 1 minute). Supernatant was removed by suction, 1 mL of Accutase was added to precipitated cell clusters, and the resultant mixture was incubated at 37° C. for 5 to 10 minutes. Several times of pipetting were performed by a pipette tip for detachment of cells, then, an equivalent amount of StemFit medium was added, and the resultant mixture was centrifuged at 200 g for 2 minutes. Supernatant was removed by suction, precipitate was suspended by addition of 1 mL of StemFit medium (comprising 10 M of Y-27632), and the number of cells was counted.

Cells were suspended in StemFit medium (comprising 10 μM of Y-27632, and 0.5 μg/cm² of iMatrix-511 Silk) so as to provide medium with 1×10⁵ cells/well/1 mL, seeded into each well of a 24-well plate, and cultured for 3 hours under conditions: 37° C. and 5% CO₂. The medium was removed from the 24-well plate and washing was performed with 1 mL of PBS. After removal of PBS, 500 μL of PFA liquid (PBS comprising 4% paraformaldehyde) was charged into each well and the plate was left to stand at room temperature for 10 minutes to fix cells. The PFA liquid was removed, 500 μL of 0.1% TritonX-100/PBS solution was added to each well, and cells were incubated on a shaker for 10 minutes (this step was not performed only for wells for detection of SSEA-4). 0.1% TritonX-100/PBS solution was discarded, 500 μL of PBS was added to each well; the plate was placed on a shaker for 5 minutes, and PBS was removed (washing operation). Washing was performed further 3 times.

After PBS was removed, 500 μL of 0.1% blocking buffer (PBS comprising 0.1% BSA) was added to each well, the plate was placed on a shaker for 30 to 60 minutes, and blocking was performed. After blocking, 500 μL of a primary antibody solution described blow was added and cells were left to stand at 4° C. overnight, allowing antibodies to react. Primary antibodies were diluted with PBS comprising 0.1% BSA as described below.
Mouse anti-human OCT3/4 antibody (Santa Cruz Biotechnology, #sc-5279, 1/400 dilution)
Rabbit anti-human NANOG antibody (REPROCELL Inc., #RCAB004P—F, 1/400 dilution)
APC-labeled rat anti-human SSEA antibody (Biolegend Inc., #330418, 1/100 dilution)

At the time of reaction with antibodies, the plate was covered with an aluminum foil for shading.

Under the shading condition, washing operation with PBS was performed 4 times. After the end of washing operation, 500 μL of secondary antibody solution described blow was added to each well (this step was not performed for wells for SSEA-4 staining, but PBS was charged), and incubation was performed on a shaker for 1 to 2 hours at room temperature under the shading condition.
Donkey anti-Mouse IgG (H+L) antibody (Highly Cross-Absorbed Secondary Antibody, Alexa Fluor 488 (Thermo Fisher Scientific Inc., A-21202))
Donkey anti-Rabbit IgG (DyLight™ 488, Biolegend Inc., #406404)

Under the shading condition, washing operation with PBS was performed 4 times, and one drop of DAPI solution (Fluoro-KEEPER Antifade Reagent, Non-Hardening Type with DAPI (NACALAI TESQUE, INC., #12745-74)) was provided to each well. Whether there are positive cells for each marker protein by nuclear stain or immunostaining using DAPI was observed by a fluorescence microscope (Keyence Corporation, BZ—X810).

Among suspension culture cells on day 57 of culture, the positive rate of each undifferentiation marker (OCT4 positive rate, NANOG positive rate and SSEA positive rate to DAPI-positive cells) was calculated; and as a result, all of the positive rates were very high as shown in Table 1.

TABLE 1

| | |
|---|---|
| OCT4 positive rate (%) | 98 |
| NANOG positive rate (%) | 96 |
| SSEA4 positive rate (%) | 94 |

### (1-4-3) Evaluation of Pluripotency by Formation of Germ Layer of Cell

### (1-4-3-1) Formation of Germ Layer

For part of suspension culture cells on day 57 of culture, the differentiation capacity into three germ layers was evaluated. Suspension culture cells were transferred to a 15-mL tube and centrifuged (200 g, for 1 minute). Supernatant was removed by suction, and precipitated cell clusters were washed by addition of 5 mL of PBS and further centrifuged (200 g, for 1 minute). Supernatant was removed by suction, 1 mL of Accutase was added to precipitated cell clusters and the mixture was incubated at 37° C. for 5 to 10 minutes. Several times of pipetting were performed by a pipette tip for detachment of cells, and an equivalent amount of StemFit medium was added and the mixture was centrifuged at 200 g for 2 minutes. Supernatant was removed by suction, precipitate was suspended by addition of 1 mL of StemFit medium (comprising 10 M of Y-27632), and the number of cells was counted.

Cells were suspended such that the number of cells was adjusted to 1.2×10⁶ cells/12 mL StemFit medium (comprising 10 μM of Y-27632) in a 15-mL tube. The entire of the cell suspension was transferred to a reservoir (AS ONE Corporation, #2-7844-02), and 100 μL thereof was dispensed into each well in the V-shaped bottom 96-well plate for suspension culture (the number of cells: 1.0×10⁴ cells/well). After the plate was centrifuged at 200 g for 3 minutes, culture was started at 37° C. and 5% CO₂. On day 2 of culture, 100 μL/well of EB medium (DMEM+10% FBS+1% sodium pyruvate+1%, penicillin/streptomycin) was added to a culture plate. On day 4 and 6 of culture, 100 μL/well of medium was exchanged.

From day 8 of culture, adherent culture was started. 500 μL of gelatin solution (0.1 w/v % solution, FUJIFILM Wako Pure Chemical Corporation, #190-15805) was charged into 4 wells of a 12-well plate, and the plate was left to stand at 37° C. for about 1 hour; and thereafter, the gelatin solution was removed, so that a gelatine-coated plate was prepared. Cells cultured as described above in the EB medium were collected from each well, and 12 to 24 cells thereof were transferred to each well of a 12-well plate. Culture was started under conditions: 37° C. and 5% CO₂. Medium exchange was performed with 1 mL of EB medium every 2 days (day 10, 12, 14, . . . ). Around day 20 from the start of culture, differentiation of germ layers was evaluated by an immunostaining method.

(1-4-3-2) Evaluation of Differentiation into Three Germ Layers (Immunostaining Method)

EB medium was removed from the 12-well plate, and the plate was washed with 2 mL of PBS. After removal of PBS, 500 μL of PFA solution was charged into each well and fixation was performed at room temperature for 10 minutes. PFA solution was removed; 1 mL of 0.1% TritonX-100/PBS solution was added to each well, and incubation was performed on a shaker for 10 minutes. 0.1% TritonX-100/PBS solution was removed, 1 mL of PBS was added to each well, the plate was placed on a shaker for 5 minutes, and then, the solution was removed (washing operation). After 1 to 2 hours later, washing operation was performed 4 times, 500 μL of 0.1% blocking buffer was added to each well, and incubation was performed on a shaker for 30 to 60 minutes (blocking).

After the end of blocking, 500 μL of primary antibody solution described below was added to each well. The primary antibody solution was diluted with 0.1 BSA/PBS solution to have the following concentration. The plate was sealed with Parafilm and left to stand overnight at 4° C.

For detection of endodermal marker TUJ1: (Mouse anti-human b-3 tubulin antibody, R&D Systems Inc., MAB1195, final concentration of 2.5 to 5 g/mL)

For detection of mesodermal marker SMA: (Mouse anti-human α-smooth muscle actin (SMA) antibody, R&D Systems Inc., MAB1420, final concentration of 2.5 to 5 g/mL)

For detection of ectodermal marker AFP: (Mouse anti-human α-fetoprotein (AFP) antibody, R&D Systems Inc., MAB1368, final concentration of 2.5 to 5 g/mL)

Washing operation with PBS was performed 4 times, and 500 μL of secondary antibody solution (which is obtained by diluting Donkey anti-Mouse IgG (H+L) antibody (Thermo Fisher Scientific Inc., A-21202) with 0.1 BSA/PBS solution by 500 to 1000 times) was added to each well. Thereafter, the plate was covered with an aluminum foil for shading. The plate was incubated on a shaker at room temperature for 1 to 2 hours, then, washing operation with PBS was performed 4 times, and one drop of DAPI solution was provided to each well. Whether there are positive cells for each marker protein by nuclear stain or immunostaining using DAPI was observed by a fluorescence microscope.

As a result, on day 23 of differentiation induction, the presence of positive cells for all of germ layer markers, TUJ1, SMA and AFP was identified in the culture cells. It was confirmed that suspension culture cells maintained the pluripotency.

### (1-5) Preservation

Thereafter, the suspension culture cells were subcultured every 4 to 5 days, cells collected at each subculture were suspended in STEM-CELLBANKER at 1×10⁶ cells/vial, and frozen stocks were prepared.

### Example 2: Establishment of iPS Cells Using Episomal Plasmid Vector

### (2-1) Culture of Blood Cell

Culture of blood cells was performed by the same method as (1-1) of Example 1.

### (2-2) Introduction of an Initialization Factor and Start of Initialization

In a 1.5-mL tube, 81.8 μL of Nucleofector solution in Amaxa™ Human CD34+Cell Nucleofector kit (Lonza Biosceince Inc., VAPA-1003), 18.2 L of Supplement solution I and 3.02 L of iPS cell Generation Episomal vector Mix (Takara Bio Inc.) were mixed, so that 103 μL of gene introduction reagent was prepared.

Mononuclear cells cultured in 2-1 were dispensed into a 15-mL tube such that each tube has 2.5×10⁶ cells. Centrifugation was performed at 200 g for 10 minutes, supernatant was removed, and 103 μL of the gene introduction reagent was added and the mixture was suspended carefully. The prepared cell/plasmid mixture solution was set to Nucleofector (Lonza Bioscience Inc.) and electroporation was performed. After electroporation, the cell suspension was swiftly moved by a dropper provided with the kit from a cuvette to a 1.5-mL tube having mononuclear culture medium and mixed such that the total amount became 1000 μL. The electroporated cell suspension was seeded such that one well had about 5×10⁵ cells.

The cell suspension was seeded into 2 wells of a 6-well plate having 4 mL/well of mononuclear culture medium, and rotational culture (90 rpm) was started under conditions: 37° C. and 5% CO₂. On day 3, 5 and 7 of culture, 2.0 mL of StemFit medium or StemFit medium+Ly-333531 (1 μM)+IWR1-endo (10 μM) was added. On day 9 of culture, the entire medium was exchanged (4 mL/well). On day 11, 13 and 15 of culture, half of the medium was exchanged (2 mL/well) and the culture was continued. Thereafter, medium exchange was performed every 4 to 5 days.

### (2-3) Subculture (Initialization Culture)

A suspension culture sample was transferred to a 15-mL tube and centrifuged (200 g, for 1 minute). Precipitated was washed with 5 mL of PBS and centrifuged (200 g, for 1 minute). Precipitated was suspended again with Accutase and incubated at 37° C. for 5 to 10 minutes. After several times of pipetting were performed with a pipette tip, an equivalent amount of StemFit medium was added and the mixture was centrifuged at 200 g for 2 minutes. Precipitate was suspended with 1 mL of StemFit medium (comprising 10 μM of Y27632). All cells were suspended with finally, 4 mL of StemFit medium (10 μM of Y27632) or StemFit medium+compound (1 μM of LY-333531, 10 μM of IWR1-endo and 10 μM of Y27632). Each suspension was seeded into a 6-well plate and rotational culture was continued (90 rpm, 37° C., 5% CO₂). Thereafter, half of medium was exchanged every 2 days and subculture was performed every 4 to 5 days.

On day 31 of culture, proliferation of about 2×10⁵ cells/well was confirmed and the entire thereof was seeded. On day 36 of culture, proliferation of about 2×10⁶ cells/well was confirmed, and seeded such that each well has 4×10⁵ cells. 1×10⁶ cells were subjected to flow cytometry analysis (see Example 3 described later), and TRA-1-60, SSEA-4 positive cells were sorted by cell sorting. Sorted cells were seeded in StemFit medium+compound (1 μM of LY-333531, 10 μM of IWR1-endo, and 10 μM of Y27632), and rotational culture was performed (90 rpm, 37° C., 5% CO₂). Half of medium was exchanged every 2 days, and subculture was performed every 5 to 6 days. After sorting, proliferation of about 7×10⁵ cells was confirmed at 3rd subculture, and the entire thereof was subcultured. Proliferation of about 4×10⁶ cells/well was confirmed at 4th subculture, and subculture was performed such that each well has 4×10⁵ cells. Cells collected at each subculture were suspended in STEM-CELLBANKER with 1×10⁶ cell/vial, and frozen stocks thereof were prepared.

### (2-4) Analysis of Cell Characteristics

### (2-4-1) Evaluation of Expression of Undifferentiation Marker of Cell by Flow Cytometry Analysis, and Single Cell Sorting

### (2-4-1-1) Procedure

Cell clusters in suspension culture were transferred to a 15-mL tube and centrifuged (200 g, for 1 minute). Supernatant was removed by suction, and precipitated cell clusters were washed with 5 mL of PBS and was further centrifuged (200 g, for 1 minute). Supernatant was removed by suction, 1 mL of Accutase was added to precipitated cell clusters, and incubation was performed at 37° C. for 5 to 10 minutes. Several times of pipetting were performed by a pipette tip for detachment of cells, and an equivalent amount of StemFit medium was added and the mixture was centrifuged at 200 g for 2 minutes. Supernatant was removed by suction, precipitate was suspended by addition of 1 mL of StemFit medium (comprising 10 M of Y-27632), and the number of cells was counted.

After the number of cells was counted, 2.5×10⁵ cells were dispensed to each of three 15-mL tubes for checking (for negative control, etc.) and 1×10⁶ cells were dispensed one 15-mL tube for sorting, and they were centrifuged at 200 g for 3 minutes. After centrifugation, supernatant was discarded and 1 to 2 mL of PBS was added. After centrifugation, supernatant was discarded, 250 μL (for checking) or 1000 μL (for sorting) of flow cytometry buffer (PBS comprising 0.1% BSA and 0.5 mM EDTA) was added, and pipetting was performed by a pipette tip, so that the cells were suspended.

The following primary antibodies were added to each sample tube, and the mixtures were incubated for about 1 hour on ice with shading.
For checking
   no antibody
   SSEA-4 (APC anti-human SSEA-4 antibody: Biolegend #330418) alone (2.5 L)
   TRA-1-60 (Alexa Fluoro 488 anti-Human TRA-1-60 antibody: Biolegend #330614) alone (2.5 μL)
For sorting
   SSEA-4 (10 L)+TRA-1-60 (10 μL)
   TRA-1-60 alone (10 μL)

After incubation, 750 μL of flow cytometry buffer was added to a tube for checking and 3 mL thereof was added to a tube for sorting, and the mixtures were centrifuged at 200 g for 3 minutes. After centrifugation, supernatant was removed, 500 μL of flow cytometry buffer was added, and pipetting was performed for suspension. A cell suspension was passed through a cell strainer. Thereafter, the samples were maintained on ice with shading.

Measurement and sorting of cells in the cell suspension were performed by a flow cytometry apparatus, Cell Sorter (Sony Corporation, SH800). Cells were collected from positive fractions for SSEA-4 and TRA-1-60; and about 1×10⁵ cells were directly collected to a 15-mL tube comprising 4 mL of StemFit medium (comprising 10 μM of Y-27632, 10 μM of IWR1-endo and 1 M of LY-333531) and were transferred to a 6-well plate for suspension culture; and then, rotational culture was started (90 rpm, 37° C., 5% CO₂). Thereafter, while the state of cells was being observed, subculture is continued every 5 to 6 days, and sequentially, frozen stocks (5×10 to 1×10⁶ cells/vial were suspended in STEM-CELLBANKER) were prepared.

For single cell sorting, a 96-well plate for suspension culture comprising 200 μL of StemFit medium (comprising 10 μM ofY-27632, 10 μM of IWR1-endo and 1 M of LY-333531) in each well was prepared, and collection and direct seeding were performed under the condition of 1 cell/well. After single cell sorting, suspension culture (medium exchange, subculture) was performed under the same conditions as in (2-2) and (2-3) for about 1.5 months (about 8 times of subculture), and clones were established. After single cell sorting, with respect to cells cultured for amplification under the suspension culture condition, detection of undifferentiation markers was performed under the same condition as above.

### (2-4-1-2) Results

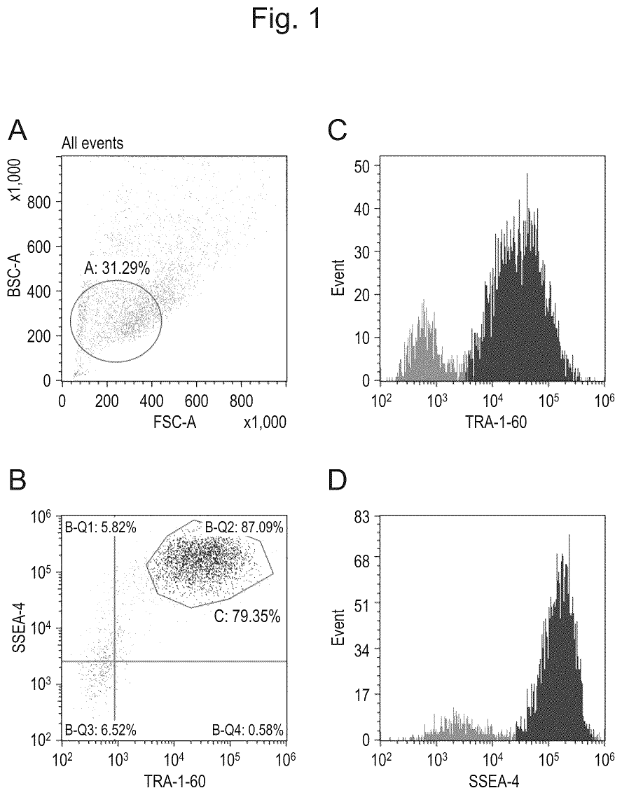

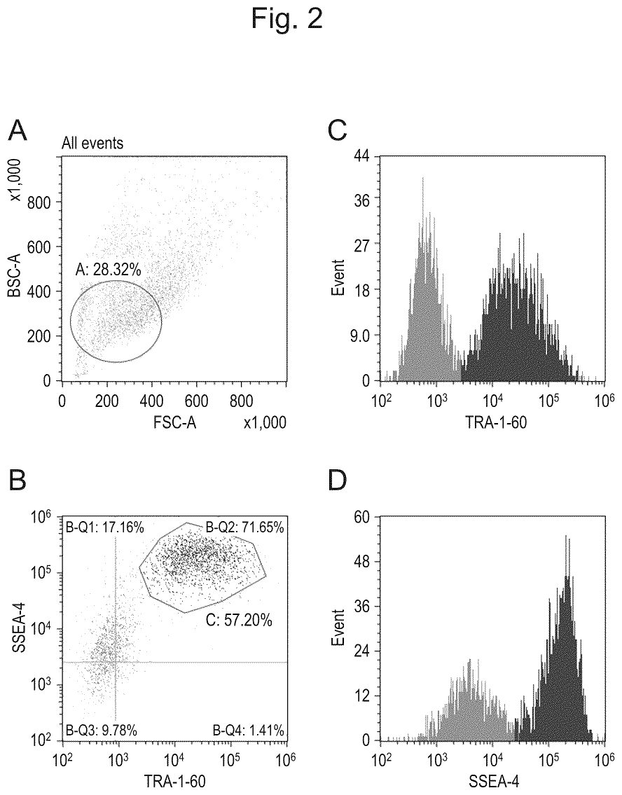

Cells after introduction of an initialization gene were cultured in suspension under each of the condition of addition of compounds (LY-333531+IWR1-endo) and the condition of addition of no compound; and cells on day 35 of culture were collected and TRA-1-60 positive cells and SSEA-4 positive cells were measured by flow cytometry. FIG. 1 shows results of flow cytometry of cells prepared by suspension culture with the condition of addition of compounds. FIG. 2 shows results of flow cytometry of cells prepared by suspension culture with the condition of addition of no compound. In both FIGS. 1 and 2, (A) shows a dot plot of side scattering light (SSC) against forward scattering light (FSC) in all events. Gating on a portion encircled by a line in the figure is performed. (B) shows a dot plot of fluorescence intensities of TRA-1-60 and fluorescence intensities of SSEA-4 of cells that are subjected to gating in (A). Cells in the range encircled by the line in the figure are sorted. (C) shows a histogram plot of fluorescence intensities of TRA-1-60 and (D) shows a histogram plot of fluorescence intensities of SSEA-4. In the condition of addition of compounds, 87% of cells were positive for TRA-1-60 and SSEA-4; while in the condition of addition of no compound, 72% of cells were positive for TRA-1-60 and SSEA-4.

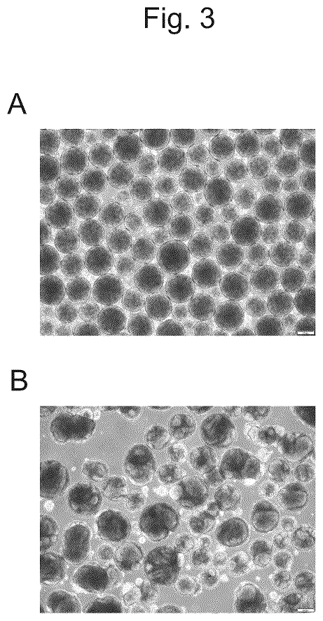

The cells on day 35 of culture were further subcultured. FIG. 3 shows microphotographs of cell aggregates in the 5th subculture prepared under the condition of addition of compounds and the condition of addition of no compound. (A) is a microphotograph of cell aggregates cultured in the condition of addition of compounds, and (B) is a microphotograph of cell aggregates cultured in the condition of addition of no compound. The figure shows that uniform cell aggregates can be obtained in cells cultured in the condition of addition of compounds. For the cells in the same passage of subculture, TRA-1-60 positive cells and SSEA-4 positive cells were measured by flow cytometry according to the same procedure as in the above (2-4-1-1); and as a result, 99% of cells were positive in the condition of addition of compounds and 39% of cells were positive in the condition of addition of no compound.

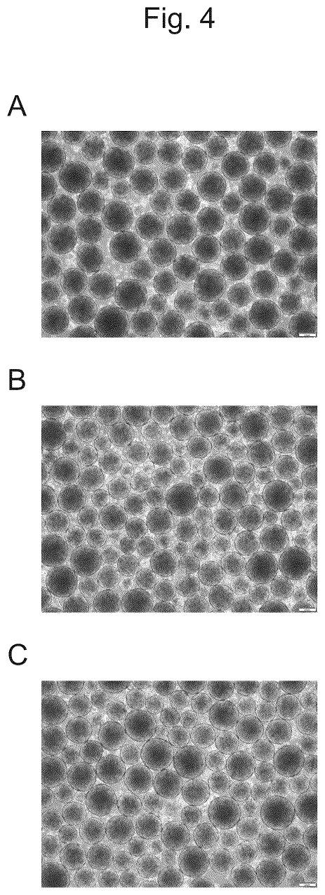

For cells on day 35 of culture, single cell sorting was performed on TRA-1-60/SSEA-4 positive cells using a cell sorter. One cell was seeded in each well of a 96-well plate, suspension culture was performed for about 1.5 months (about 8 times of subculture) in the condition of addition of compounds and the condition of addition of no compound, and 10 clones were obtained. FIG. 4 shows microphotographs of cell aggregates of suspension culture cells of 3 clones (#1, #2 and #3) of the 10 clones. (A), (B) and (C) show microphotographs for clone #1, clone #2 and clone #3, respectively. Regarding these clones #1, #2 and #3, the positive rates for TRA-1-60 were measured using flow cytometry; and as a result, all of the positive rates were very high, which were 93.66%, 94.18% and 81.99%, respectively.

### (2-4-2) Evaluation of Undifferentiation Marker Expression of Cell by Immunostaining

Using cells of 11th subculture of 3 clones (#1 to 3) sorted and cultured for amplification in 2-4-1 as the subject, immunostaining was performed by the following procedure. Suspension culture cell clusters of each clone were transferred to a 15 mL tube and centrifuged (200 g, for 1 minute). Supernatant was removed by suction, and precipitated cell clusters were washed with 5 mL of PBS and further centrifuged (200 g, for 1 minute). Supernatant was removed by suction, 1 mL of Accutase was added to precipitated cell clusters, and the resultant mixture was incubated at 37° C. for 5 to 10 minutes. Several times of pipetting were performed by a pipette tip for detachment of cells, then, an equivalent amount of StemFit medium was added, and the resultant mixture was centrifuged at 200 g for 2 minutes. Supernatant was removed by suction, precipitate was suspended by addition of 1 mL of StemFit medium (comprising 10 μM of Y-27632), and the number of cells was counted.

Cells were suspended in StemFit medium (comprising 10 M of Y-27632, and 0.5 μg/cm² of iMatrix-511 Silk) so as to provide medium with 1×10⁵ cells/well/1 mL, seeded into each well of a 24-well plate, and cultured for 3 hours under conditions: 37° C. and 5% CO². The medium was removed from the 24-well plate and washing was performed with 1 mL of PBS. After removal of PBS, 500 μL of PFA liquid (PBS comprising 4% paraformaldehyde) was charged into each well and the plate was left to stand at room temperature for 10 minutes to fix cells. The PFA liquid was removed, 500 μL of 0.1% TritonX-100/PBS solution was added to each well, and cells were incubated on a shaker for 10 minutes (this step was not performed only for wells for detection of SSEA-4). 0.1% TritonX-100/PBS solution was discarded, 500 μL of PBS was added to each well; the plate was placed on a shaker for 5 minutes, and PBS was removed (washing operation). Washing operation was performed further 3 times.

After PBS was removed, 500 μL of 0.1% blocking buffer (PBS comprising 0.1% BSA) was added to each well, the plate was placed on a shaker for 30 to 60 minutes, and blocking was performed. After blocking, 500 μL of a primary antibody solution described blow was added and cells were left to stand at 4° C. overnight, allowing antibodies to react. Primary antibodies were diluted with PBS comprising 0.1% BSA as described below.
Mouse anti-human OCT3/4 antibody (Santa Cruz Biotechnology, #sc-5279, 1/400 dilution)
Rabbit anti-human NANOG antibody (REPROCELL Inc., #RCAB004P-F, 1/400 dilution)
Primary SSEA antibody (APC anti-human SSEA-4 antibody, Biolegend Inc., #330418, 1/100 dilution)

At the time of reaction with antibodies, the plate was covered with an aluminum foil for shading.

Under the shading condition, washing operation with PBS was performed 4 times. After the end of washing operation, 500 μL of secondary antibody solution described blow was added to each well (this step was not performed for wells for SSEA-4 staining, but PBS was charged), and incubation was performed on a shaker for 1 to 2 hours at room temperature under the shading condition.
Donkey anti-Mouse IgG (H+L) antibody (Highly Cross-Absorbed Secondary Antibody, Alexa Fluor 488 (Thermo Fisher Scientific Inc., A-21202))
Donkey anti-Rabbit IgG (DyLight™ 488, Biolegend Inc., #406404)

Under the shading condition, washing operation with PBS was performed 4 times, and one drop of DAPI solution (Fluoro-KEEPER Antifade Reagent, Non-Hardening Type with DAPI (NACALAI TESQUE, INC., #12745-74)) was provided to each well. Whether there are positive cells for each marker protein by nuclear stain or immunostaining using DAPI was observed by a fluorescence microscope (Keyence Corporation, BZ—X810).

With respect to cell aggregates (cell population) obtained by culturing cells of 3 sorted clones (#1 to 3), which were initialized and cultured for amplification using episomal plasmid, the positive rate of each undifferentiation marker (OCT4 positive rate, NANOG positive rate and SSEA positive rate to DAPI-positive cells) was calculated; and as a result, all of the positive rates were very high as shown in Table 2.

TABLE 2

| | Clone | | |
|---|---|---|---|
| | #1 | #2 | #3 |
| OCT4 POSITIVE RATE (%) | 94 | 94 | 93 |
| NANOG positive rate (%) | 98 | 95 | 97 |
| SSEA4 positive rate (%) | 95 | 95 | 97 |

### (2-4-3) Evaluation of Pluripotency of Cell by Formation of Germ Layer

(2-4-3-1) Differentiation of Cell into Three Germ Layers

Cell clusters of 12th passage in suspension culture of 3 clones sorted and cultured for amplification in 2-4-1 (same as clones #1 to 3 evaluated for the expression of undifferentiation markers in 2-4-2) were transferred into a 15-mL tube and centrifuged (200 g, for 1 minute). Supernatant was removed by suction, and precipitated cell clusters were washed by addition of 5 mL of PBS and further centrifuged (200 g, for 1 minute). Supernatant was removed by suction, 1 mL of Accutase was added to precipitated cell clusters and the mixture was incubated at 37° C. for 5 to 10 minutes. Several times of pipetting were performed by a pipette tip for detachment of cells, and an equivalent amount of StemFit medium was added and the mixture was centrifuged at 200 g for 2 minutes. Supernatant was removed by suction, precipitate was suspended by addition of 1 mL of StemFit medium (comprising 10 M of Y-27632), and the number of cells was counted.

Cells were suspended such that the number of cells was adjusted to 1.2×10⁶ cells/12 mL StemFit medium (comprising 10 μM of Y-27632) in a 15-mL tube. The entire of the cell suspension was transferred to a reservoir (AS ONE Corporation, #2-7844-02), and 100 μL thereof was dispensed into each well in the V-shaped bottom 96-well plate for suspension culture (the number of cells: 1.0×10⁴ cells/well). After the plate was centrifuged at 200 g for 3 minutes, culture was started at 37° C. and 5% CO₂. On day 2 of culture, 100 μL/well of EB medium (DMEM+10%, FBS+1%, sodium pyruvate+1%, penicillin/streptomycin) was added to a culture plate. On day 4 and 6 of culture, 100 μL/well of medium was exchanged.

From day 8 of culture, adherent culture was started. 500 μL of gelatin solution (0.1 w/v % solution, FUJIFILM Wako Pure Chemical Corporation, #190-15805) was charged into 4 wells of a 12-well plate, and the plate was left to stand at 37° C. for about 1 hour; and thereafter, the gelatin solution was removed, so that a gelatine-coated plate was prepared. Cells cultured as described above in the EB medium were collected from each well, and 12 to 24 cells thereof were transferred to each well of a 12-well plate. Culture was started under conditions: 37° C. and 5% CO₂. On every 2 days (day 10, 12, 14, . . . ), medium exchange was performed with 1 mL of EB medium. Around day 20 from the start of culture, differentiation of germ layers was evaluated by an immunostaining method.

(2-4-3-2) Evaluation of Differentiation into Three Germ Layers (Immunostaining Method)

EB medium was removed from the 12-well plate, and the plate was washed with 2 mL of PBS. After removal of PBS, 500 μL of PFA solution was charged into each well and fixation was performed at room temperature for 10 minutes. PFA solution was removed; 1 mL of 0.1% TritonX-100/PBS solution was added to each well, and incubation was performed on a shaker for 10 minutes. 0.1% TritonX-100/PBS solution was removed, 1 mL of PBS was added to each well, the plate was placed on a shaker for 5 minutes, and then, the solution was removed (washing operation). After 1 to 2 hours later, washing operation was performed 4 times, 500 μL of 0.1% blocking buffer was added to each well, and incubation was performed on a shaker for 30 to 60 minutes (blocking).

After the end of blocking, 500 μL of primary antibody solution described below was added to each well. The primary antibody solution was diluted with 0.1 BSA/PBS solution to have the following concentration. The plate was sealed with Parafilm and left to stand overnight at 4° C.

For detection of endodermal marker TUJ1: (Mouse anti-human b-3 tubulin antibody, R&D Systems Inc., MAB1195, final concentration of 2.5 to 5 μg/mL)

For detection of mesodermal marker SMA: (Mouse anti-human α-smooth muscle actin (SMA) antibody, R&D Systems Inc., MAB1420, final concentration of 2.5 to 5 μg/mL)

For detection of ectodermal marker AFP: (Mouse anti-human α-fetoprotein (AFP) antibody, R&D Systems Inc., MAB1368, final concentration of 2.5 to 5 μg/mL)

Washing operation with PBS was performed 4 times, and 500 μL of secondary antibody solution (which is obtained by diluting Donkey anti-Mouse IgG (H+L) antibody (Thermo Fisher Scientific Inc., A-21202) with 0.1 BSA/PBS solution by 500 to 1000 times) was added to each well. Thereafter, the plate was covered with an aluminum foil for shading. The plate was incubated on a shaker at room temperature for 1 to 2 hours, then, washing operation with PBS was performed 4 times, and one drop of DAPI solution was provided to each well. Whether there are positive cells for each marker protein by nuclear stain or immunostaining using DAPI was observed by a fluorescence microscope.

In cell populations of 3 clones, the presence of all of TUJ1, SMA and AFP positive cells was confirmed. It was confirmed that these clones all maintained the pluripotency.

All publications, patents and patent applications cited herein are incorporated herein by reference in their entirety.

1. A method for producing an induced pluripotent stem cell (iPS cell), comprising the following steps I and II:
I. introducing an initialization gene into a somatic cell; and
II. performing initialization and amplification culture of the cell into which the gene has been introduced, in a liquid medium comprising at least one selected from the group consisting of a protein kinase Cβ (PKCβ) inhibitor and a WNT inhibitor under a suspension culture condition.

2. The method according to claim 1, further comprising the following steps III and IV:
III. sorting an undifferentiation marker positive cell from the cell obtained in the step II; and
IV. performing suspension culture of the undifferentiation marker positive cell sorted in the step III to obtain a cell population.

3. The method according to claim 2, wherein the step III is a step of fractionating the sorted cell.

4. The method according to claim 2, wherein the undifferentiation marker is at least one selected from the group consisting of SSEA-4, TRA-1-60, TRA-1-81 and TRA-2-49.

5. The method according to claim 1, wherein the somatic cell is a blood mononuclear cell.

6. The method according to claim 1, wherein the gene to be introduced into the somatic cell is incorporated into an episomal plasmid or Sendai virus and introduced into the somatic cell.

7. The method according to claim 1, wherein the initialization gene to be introduced comprises at least one gene selected from the group consisting of an OCT3/4 gene, a KLF4 gene and an L-MYC gene.

8. The method according to claim 1, wherein the liquid medium used in and after the step II comprises at least one selected from L-ascorbic acid, insulin, transferrin, selenium and sodium hydrogen carbonate.

9. The method according to claim 1, wherein the liquid medium used in and after the step II comprises FGF2 and/or TGF-β1.

10. The method according to claim 1, wherein the liquid medium used in and after the step II comprises a ROCK inhibitor.

11. The method according to claim 10, wherein the ROCK inhibitor is Y-27632.

12. The method according to claim 2, wherein, in the cell population obtained in the step IV, the proportion of cells positive for OCT4 is 90% or higher, and the proportion of cells positive for NANOG in the obtained cell population is 90% or higher.

13. The method according to claim 1, wherein the step II comprises a procedure of colleting a cell aggregate.

14. The method according to claim 2, wherein the step IV comprises a procedure of collecting a cell aggregate.

15. A cell population comprising iPS cells produced by the method according to claim 1.

16-23. (canceled)
